(19) United States
(12) Patent Application Publication
(10) Pub. No.: US 2025/0329467 A1
(43) Pub. Date: Oct. 23, 2025

(54) WORKFLOWS FOR DISCOVERY AND DEPLOYMENT OF DIAGNOSTIC ASSAYS COMBINING PROTEOMIC AND GENOMIC INFORMATION

(71) Applicant: DELFI DIAGNOSTICS, INC., Baltimore, MD (US)

(72) Inventors: Jacob Carey, Baltimore, MD (US); Joseph Catallini, Baltimore, MD (US); Nicholas Dracopoli, Baltimore, MD (US); Keith Lumbard, Baltimore, MD (US); Timothy McDaniel, Baltimore, MD (US); Jamie Medina, Baltimore, MD (US); Kathryn Todd, Menlo Park, CA (US); Bridget Tripp, Baltimore, MD (US)

(21) Appl. No.: 19/187,960

(22) Filed: Apr. 23, 2025

Related U.S. Application Data
(60) Provisional application No. 63/637,831, filed on Apr. 23, 2024.

(51) Int. Cl. G16H 50/30; C12Q 1/6869; C12Q 1/6886; G01N 33/574; G01N 33/68; G01N 35/00; G01N 35/10; G06N 20/00; G16B 25/10; G16B 40/10; G16B 40/20; G16H 10/40; G16H 50/70; G16H 70/60

(52) U.S. Cl. CPC G16H 50/30; C12Q 1/6869; C12Q 1/6886; G01N 33/57423; G01N 33/6893; G01N 35/0099; G01N 35/10; G06N 20/00; G16B 25/10; G16B 40/10; G16B 40/20; G16H 10/40; G16H 50/70; G16H 70/60

(57) ABSTRACT

This present disclosure provides an integrated workflow and systems for the efficient deployment of integrated genomic and proteomic diagnostic assays. The diagnostic assays include a proteomic component, a genetic component, liquid handling robots, a LIMS system, and a software classifier component. Also provided herein are systems and diagnostic assays for the detection of lung cancer.

### CROSS REFERENCE TO RELATED APPLICATIONS

This application claims the benefit of priority under U.S.C. § 119 (e) of U.S. Provisional Patent Application Ser. No. 63/637,831 filed on Apr. 23, 2024, the contents of which are herein incorporated by reference in its entirety.

### FIELD OF THE INVENTION

The invention relates generally to workflows that utilize genetic analysis and more specifically to methods and systems for analysis of cell-free DNA fragment size densities in conjunction with proteomic analysis to detect and/or assess disease in a subject.

### BACKGROUND OF THE INVENTION

When it comes to detecting disease and pinpointing the right treatment for each patient, timing can be crucial. If a disease is diagnosed early and accurately, progression may be slowed or even stopped and the possibility of cure increases. Diagnostic testing can not only arm patients, families and healthcare professionals with information that may lead to the best possible outcome, it can improve health system efficiency. There is an unmet clinical need for the development of non-invasive approaches to improve disease screening for high-risk individuals and ultimately the general population.

Diagnostic assay systems that integrate genomic and proteomic information offers a more comprehensive understanding of diseases, potentially leading to earlier and more accurate diagnoses. This approach combines the power of genomic analysis with the insights gained from proteomic analysis, providing a richer picture of biological processes at play in a disease.

### SUMMARY OF THE INVENTION

The present disclosure provides a diagnostic assay system. In one aspect, the diagnostic assay system includes a genomic component, a proteomic component, a liquid handling robot configured to carry out one or more assay steps of the genomic and/proteomic components, a laboratory information management system (LIMS), and a software classifier component.

In one aspect, the genomic component is configured to a. generate DNA sequences from input patient samples using a next-generation sequencing (NGS)-based assay workflow; b. associate DNA sequencing results with source patients using DNA-based barcodes; and c. process DNA sequencing results associated with each patient through a computer analysis pipeline. In one aspect, the proteomic component is configured to a. perform a multiplexed protein detection assay with an NGS-based readout; b. multiplex a range of proteins from a handful to tens of thousands in a single sample; c. target specific protein content with a cocktail of chosen affinity binding molecules; d. associate NGS readout of protein assay results with source patients using DNA-based barcodes compatible with the genomic component; and e. process NGS readout of protein assay results associated with each patient through a computer analysis pipeline. In one aspect, laboratory information management system (LIMS) configured to: a. track one or more assay steps; b. govern actions of the liquid handling robots; c. track and enforce the use of any protein-content specifying reagent at the appropriate point in the assay based on operator selection or a test requisition form; and d. track patient identities or patient-associated codes for samples and generated test information for both the proteomic and genomic components. In one aspect, the software classifier component is configured to combine information generated by the genomic and proteomic components into a reported risk score for a patient for one or more types of cancer.

In one aspect, the diagnostic assay system further includes a pooling feature configured to pool NGS libraries from the genomic and proteomic components to allow simultaneous readout of both components. In one aspect, the proteomic component includes a modular protein content design, which includes two or more disease-specific associated protein reagents, enabling a laboratory to run multiple tests simultaneously on the same robot deck with each test having differences in protein reagent, classifier or both, and reporting among the different disease tests. In one aspect, the proteomic component includes a universal protein content design, comprising: a single protein reagent containing all affinity binding molecules for all tests, with differentiation of employed content for different tests occurring informatically through filtering of sequences associated with specific proteins, followed by the use of disease-specific classifiers and reports. In one aspect, the proteomic discovery system includes the genomic component of the assay system; the proteomic component of the assay system using a large discovery panel of protein content; one or more cohorts of patients known to have the disease or diseases in question; the running of the proteomic component of the assay system with a large discovery panel of protein content; and a machine learning algorithm configured to generate a classifier that combines information generated by the genomic and proteomic components into a reported risk score for a patient for the disease or diseases in question.

In one aspect, the proteomic component is further configured to allow for the discovery and efficient deployment of integrated genomic and proteomic diagnostic assays, enabling efficient discovery and modular deployment of protein-based panels in the context of a genomic-based workflow.

In one embodiment, the present disclosure provides a method for detecting lung cancer in an individual. In one aspect, the method includes, a. analyzing a sample obtained from the individual to detect a presence of a panel of proteins; b. assessing cell-free DNA fragmentation patterns in the sample; c. applying a machine learning model to the detected proteins and cell-free DNA fragmentation patterns to generate an area under the curve (AUC) score; and d. determining the presence of lung cancer in the individual based on the AUC score. In one aspect, the sample is a L101 sample. In one aspect, the machine learning model includes a gradient boosting machine (GBM) model. In one aspect, the AUC score for the combined analysis of proteins and cell-free DNA fragmentation patterns is at least about 0.90. In one aspect, the AUC score for stage I lung cancer is at least about 0.81. In one aspect, the method further includes (a) evaluating the performance of the combined protein and cell-free DNA fragmentation model at 50% specificity; and (b) determining the sensitivity for detecting stage I, stage II, and stage III & IV lung cancer. In one aspect, the sensitivity for detecting stage I lung cancer is at least about 88%. In one aspect, the sensitivity for detecting stage II lung cancer is at least about 96%. In one aspect, the sensitivity for detecting stage III & IV lung cancer is about 100%. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

In one embodiment, the present disclosure provides a method for detecting lung cancer in an individual. In one aspect, the method includes a. a protein platform configured to analyze a sample from the individual to detect a presence of a panel of; b. a cell-free DNA fragmentation analysis module configured to assess cell-free DNA fragmentation patterns in the sample; c. a machine learning module configured to apply a machine learning model to the detected proteins and cell-free DNA fragmentation patterns to generate an AUC score; and d. a diagnostic module configured to determine the presence of lung cancer in the individual based on the AUC score. In one aspect, the machine learning module includes a gradient boosting machine (GBM) model. In one aspect, the diagnostic module is further configured to evaluate the performance of the combined protein and cell-free DNA fragmentation model at about 50% specificity and to determine the sensitivity for detecting stage I, stage II, and stage III & IV lung cancer. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

In one embodiment, the present disclosure provides a non-transitory computer-readable medium storing instructions that, when executed by a processor, cause the processor to perform a method for detecting lung cancer in an individual. In one aspect, the method includes a. receiving data indicative of a presence of a panel of proteins in a sample from the individual, wherein the proteins are analyzed using a protein platform; b. receiving data indicative of cell-free DNA fragmentation patterns in the sample; c. applying a machine learning model to the received data to generate an AUC score; and d. outputting a determination of the presence of lung cancer in the individual based on the AUC score. In one aspect, the machine learning model includes a gradient boosting machine (GBM) model. In one aspect, the method further includes instructions for evaluating the performance of the combined protein and cell-free DNA fragmentation model at about 50% specificity and for determining the sensitivity for detecting stage I, stage II, and stage III & IV lung cancer. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

In one embodiment, the present disclosure provides a method for detecting lung cancer in an individual. In one aspect, the method includes a. measuring levels of a panel of literature-curated proteins in a sample from the individual using a protein platform; b. analyzing cell-free DNA fragmentation patterns in the sample; c. applying a machine learning model to the measured levels of the proteins and the analyzed cell-free DNA fragmentation patterns to determine a combined area under the curve (AUC) score; and d. diagnosing the presence or stage of lung cancer in the individual based on the combined AUC score. In one aspect, the sample is a L101 sample. In one aspect, machine learning model is a gradient boosting machine (GBM) model. In one aspect, the panel of proteins is associated with lung cancer risk. In one aspect, the machine learning model provides a combined AUC of about 0.86 (0.82-0.9) for the proteins. In one aspect, the combined AUC for stage I lung cancer is about 0.75 (0.68-0.82) when using the proteins alone. In one aspect, the combined AUC for detecting lung cancer using both proteins and cell-free DNA fragmentation is about 0.90 (0.87-0.93). In one aspect, combined AUC for stage I lung cancer using both proteins and cell-free DNA fragmentation is about 0.81 (0.75-0.88). In one aspect, the method includes evaluating the performance of the combined protein and cell-free DNA fragmentation model at about 50% specificity to determine sensitivities for different stages of lung cancer. In one aspect, the sensitivities at about 50% specificity are about 88% for stage I, about 96% for stage II, and about 100% for stages III & IV. In one aspect, the method further includes identifying a subset of proteins from the panel of proteins that contribute to detection benefit. In one aspect, the identification of the subset of proteins is performed using an iterative process that removes the least influential protein in each iteration. In one aspect, the iterative process results in a list of top influential proteins that maximizes performance and lowers the potential cost of the combined assay. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

The present disclosure provides a system for detecting lung cancer in an individual. In one aspect, the system includes a. a protein platform configured to measure levels of a panel of literature-curated proteins in a sample from the individual; b. an analyzer configured to analyze cell-free DNA fragmentation patterns in the sample; c. a processor configured to apply a machine learning model to the measured levels of the proteins and the analyzed cell-free DNA fragmentation patterns to determine a combined AUC score; and. d diagnostic module configured to diagnose the presence or stage of lung cancer in the individual based on the combined AUC score. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

The present disclosure also provides a non-transitory computer-readable medium containing instructions that, when executed by a processor, perform a method for detecting lung cancer in an individual. In one aspect, the computer-readable medium includes a. receiving data corresponding to levels of a panel of literature-curated proteins measured in a sample from the individual; b. receiving data corresponding to cell-free DNA fragmentation patterns analyzed in the sample; c. applying a machine learning model to the received data to determine a combined AUC score; and d. outputting a diagnosis for the presence or stage of lung cancer in the individual based on the combined AUC score.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1 depicts an assay system with modular protein reagent design.

FIG. 2 depicts an assay system with a universal protein reagent design.

FIG. 3 depicts the development of a disease-specific panel for use in a modular protein reagent design.

FIG. 4 depicts the performance of top 6 individual proteins (See also Table 2).

FIG. 5 depicts the performance of a combined protein GBM model.

FIG. 6 depicts the performance of a combined protein and cell-free DNA fragmentation approach.

FIG. 7 depicts the performance of a combined protein and cell-free DNA fragmentation approach in stage I lung cancer individuals.

FIG. 8 depicts the approach for identifying proteins that greatly contribute detection benefit to potentially reducing 100 proteins to a list of 20 or less, for potential reduction in cost of an assay including both fragmentation and proteins.

FIG. 9 depicts the model performance following schema in FIG. 8, where the least influential protein is removed in each iteration. The top curve represents AUC of stacked scores (Fra. Protein GBM and the bottom curve represents AUC of protein-only GBM scores.

FIG. 10 depicts a list of top influential proteins after following the schema in FIG. 8 (See also Table 3).

FIG. 11 depicts a sensitivity analysis comparing fragmentation alone, or in combination with protein panels. In FIG. 11, the first whisker represent fragmentation only, whereas the second and third whisker for each repeat represent fragmentation plus protein panel.

FIG. 12 depicts an analysis of sensitivity and specificity for fragmentation alone compared with fragmentation and protein panel. The curve to the left is fragmentation plus protein panel whereas the curve to the right is fragmentation only.

### DETAILED DESCRIPTION

This disclosed invention entails an integrated workflow and system for the discovery and efficient deployment of integrated genomic and proteomic diagnostic assays. It allows for efficient discovery and modular deployment of protein-based panels in the context of a genomic based workflow. Throughout this disclosure, “genomic” is used generally to mean comprehensive analysis of DNA sequencing and could include various analytic approaches of DNA sequencing information including mutational, copy number, mitochondrial DNA, or fragmentomic analysis.

The addition of protein signals to a genomic assay is expected to improve the diagnostic performance compared to genomics alone, and certain features of this system allow such addition to be deployable in a cost-effective and minimally disruptive way. Also, aspects of the system allow for the same workflow to allow multiple diagnostic tests to be run in the same laboratory with the identical workflow, or, in some instances, with the identical workflow with the exception of a single reagent that specifies protein content. The ability to use a unified workflow in discovery and deployment, across different diseases and analytes will generate efficiencies throughout the lab by reducing training, reagent inventories, documentation, spare parts, number of instruments, and time needed for new test development.

In some aspects, the assay system includes the following components: a genomic component, a proteomic component, a liquid handling robot configured to carry out one or more steps of the genomic and/or proteomic components, a laboratory information management system (LIMS), and/or a software classifier component configured to combine information generated by the genomic and proteomic components into a reported risk score for a patient for one or more cancer types.

In some aspects, the genomic component includes a) a next-generation sequencing (NGS)-based assay workflow that generates DNA sequences from input patient samples. b) DNA-based barcodes that allow for the association between DNA sequencing results and the source patient. c) a computer analysis pipeline that processes DNA sequencing results associated with each patient.

In some aspects, the NGS can be whole genome sequencing (WGS), whole exome sequencing (WES), targeted sequencing, methylation sequencing, cell-free (cfDNA) sequencing and/or targeted sequencing. In some aspects, cfDNA from an individual (e.g., an individual having, or suspected of having, cancer) can be processed into sequencing libraries which can be subjected to whole genome sequencing (e.g., low-coverage whole genome sequencing), mapped to the genome, and analyzed to determine cfDNA fragment lengths. In some aspects, mapped sequences are analyzed in non-overlapping windows covering the genome. In some aspects, windows can be any appropriate size. In some aspects, the windows are from thousands to millions of bases in length. As one non-limiting example, a window can be about 5 megabases (Mb) long. Any appropriate number of windows can be mapped. For example, tens to thousands of windows can be mapped in the genome. For example, hundreds to thousands of windows can be mapped in the genome. A cfDNA fragmentation profile can be determined within each window.

In some aspects, a sequencing “library” is created from the sample. The DNA (or cDNA) sample is processed into relatively short double-stranded fragments (100-800 bp). Depending on the specific application, DNA fragmentation can be performed in a variety of ways, including physical shearing, enzyme digestion, and PCR-based amplification of specific genetic regions. In some aspects, the resulting DNA fragments are ligated to technology-specific adaptor sequences, forming a fragment library. These adaptors may also have a unique molecular “barcode”, so each sample can be tagged with a unique DNA sequence. This allows for multiple samples to be mixed together and sequenced at the same time. For example, barcodes 1-20 can be used to individually label 20 samples and then analyze them in a single sequencing run.

The present disclosure provides a diagnostic assay system. In one aspect, the diagnostic assay system includes a genomic component, a proteomic component, a liquid handling robots configured to carry out one or more assay steps of the genomic and/proteomic components, a laboratory information management system (LIMS), and a software classifier component.

In one aspect, the genomic component is configured to a. generate DNA sequences from input patient samples using a next-generation sequencing (NGS)-based assay workflow; b. associate DNA sequencing results with source patients using DNA-based barcodes; and c. process DNA sequencing results associated with each patient through a computer analysis pipeline. In one aspect, the proteomic component is configured to a. perform a multiplexed protein detection assay with an NGS-based readout; b. multiplex a range of proteins from a handful to tens of thousands in a single sample; c. target specific protein content with a cocktail of chosen affinity binding molecules; d. associate NGS readout of protein assay results with source patients using DNA-based barcodes compatible with the genomic component; and e. process NGS readout of protein assay results associated with each patient through a computer analysis pipeline. In one aspect, the laboratory information management system (LIMS) configured to: a. track one or more assay steps; b. govern actions of the liquid handling robots; c. track and enforce the use of any protein-content specifying reagent at the appropriate point in the assay based on operator selection or a test requisition form; and d. track patient identities or patient-associated codes for samples and generated test information for both the proteomic and genomic components. In one aspect, the software classifier component is configured to combine information generated by the genomic and proteomic components into a reported risk score for a patient for one or more types of cancer.

In one aspect, the diagnostic assay system, further includes a pooling feature configured to pool NGS libraries from the genomic and proteomic components to allow simultaneous readout of both components. In one aspect, the proteomic component includes a modular protein content design, which includes two or more disease-specific associated protein reagents, enabling a laboratory to run multiple tests simultaneously (in parallel) on the same robot deck with each test having differences in protein reagent, classifier or both; and also allowing the reporting among the different disease tests. In one aspect, the proteomic component includes a universal protein content design, comprising: a single protein reagent containing all affinity binding molecules for all tests, with differentiation of employed content for different tests occurring informatically through filtering of sequences associated with specific proteins, followed by the use of disease-specific classifiers and reports. In one aspect, the proteomic discovery system includes the genomic component of the assay system; the proteomic component of the assay system using a large discovery panel of protein content; one or more cohorts of patients known to have the disease or diseases in question; the running of the proteomic component of the assay system with a large discovery panel of protein content; and a machine learning algorithm configured to generate a classifier that combines information generated by the genomic and proteomic components into a reported risk score for a patient for the disease or diseases in question.

In one aspect, the proteomic component is further configured to allow for the discovery and efficient deployment of integrated genomic and proteomic diagnostic assays, enabling efficient discovery and modular deployment of protein-based panels in the context of a genomic-based workflow.

In one embodiment, the present disclosure provides a method for detecting lung cancer in an individual. In one aspect, the method includes, a. obtaining a sample from the individual; b. analyzing the sample to detect a presence of a panel of proteins; c. assessing cell-free DNA fragmentation patterns in the sample; d. applying a machine learning model to the detected proteins and cell-free DNA fragmentation patterns to generate an area under the curve (AUC) score; and e. determining the presence of lung cancer in the individual based on the AUC score. In one aspect, the sample is a L101 sample. In one aspect, the machine learning model includes a gradient boosting machine (GBM) model. In one aspect, the AUC score for the combined analysis of proteins and cell-free DNA fragmentation patterns is at least about 0.90. In one aspect, the AUC score for stage I lung cancer is at least about 0.81. In one aspect, the method further includes (a) evaluating the performance of the combined protein and cell-free DNA fragmentation model at 50% specificity; and (b) determining the sensitivity for detecting stage I, stage II, and stage III & IV lung cancer. In one aspect, the sensitivity for detecting stage I lung cancer is at least about 88%. In one aspect, the sensitivity for detecting stage II lung cancer is at least about 96%. In one aspect, the sensitivity for detecting stage III & IV lung cancer is about 100%. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

In one embodiment, the present disclosure provides a method for detecting lung cancer in an individual. In one aspect, the method includes a. a protein platform configured to analyze a sample from the individual to detect a presence of a panel of; b. a cell-free DNA fragmentation analysis module configured to assess cell-free DNA fragmentation patterns in the sample; c. a machine learning module configured to apply a machine learning model to the detected proteins and cell-free DNA fragmentation patterns to generate an AUC score; and d. a diagnostic module configured to determine the presence of lung cancer in the individual based on the AUC score. In one aspect, the machine learning module includes a gradient boosting machine (GBM) model. In one aspect, the diagnostic module is further configured to evaluate the performance of the combined protein and cell-free DNA fragmentation model at about 50% specificity and to determine the sensitivity for detecting stage I, stage II, and stage III & IV lung cancer. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof. In one aspect, the method includes detecting one, two, three, four, five, six, seven, eight, nine, ten, eleven, twelve, thirteen, fourteen, fifteen, twenty, thirty or more proteins in a panel. In one aspect, the one, two, three, four, five, six, seven, eight, nine, ten, eleven, twelve, thirteen, fourteen, fifteen, twenty, thirty or more proteins are selected from ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53. Any combination of proteins ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 may be used in a panel in the methods described herein.

In one embodiment, the present disclosure provides a non-transitory computer-readable medium storing instructions that, when executed by a processor, cause the processor to perform a method for detecting lung cancer in an individual. In one aspect, the method includes a. receiving data indicative of a presence of a panel of proteins in a sample from the individual, wherein the proteins are analyzed using a protein platform; b. receiving data indicative of cell-free DNA fragmentation patterns in the sample; c. applying a machine learning model to the received data to generate an AUC score; and d. outputting a determination of the presence of lung cancer in the individual based on the AUC score. In one aspect, the machine learning model includes a gradient boosting machine (GBM) model. In one aspect, the method further includes instructions for evaluating the performance of the combined protein and cell-free DNA fragmentation model at about 50% specificity and for determining the sensitivity for detecting stage I, stage II, and stage III & IV lung cancer. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof. In one aspect, the method includes detecting one, two, three, four, five, six, seven, eight, nine, ten, eleven, twelve, thirteen, fourteen, fifteen, twenty, thirty or more proteins in a panel. In one aspect, the one, two, three, four, five, six, seven, eight, nine, ten, eleven, twelve, thirteen, fourteen, fifteen, twenty, thirty or more proteins are selected from ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53. Any combination of proteins ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 may be used in a panel in the methods described herein.

In one embodiment, the present disclosure provides a method for detecting lung cancer in an individual. In one aspect, the method includes a. measuring levels of a panel of literature-curated proteins in a sample from the individual using a protein platform; b. analyzing cell-free DNA fragmentation patterns in the sample; c. applying a machine learning model to the measured levels of the proteins and the analyzed cell-free DNA fragmentation patterns to determine a combined area under the curve (AUC) score; and d. diagnosing the presence or stage of lung cancer in the individual based on the combined AUC score. In one aspect, the sample is a L101 sample. In one aspect, machine learning model is a gradient boosting machine (GBM) model. In one aspect, the panel of proteins is associated with lung cancer risk. In one aspect, the machine learning model provides a combined AUC of about 0.86 (0.82-0.9) for the proteins. In one aspect, the combined AUC for stage I lung cancer is about 0.75 (0.68-0.82) when using the proteins alone. In one aspect, the combined AUC for detecting lung cancer using both proteins and cell-free DNA fragmentation is about 0.90 (0.87-0.93). In one aspect, combined AUC for stage I lung cancer using both proteins and cell-free DNA fragmentation is about 0.81 (0.75-0.88). In one aspect, the method includes evaluating the performance of the combined protein and cell-free DNA fragmentation model at about 50% specificity to determine sensitivities for different stages of lung cancer. In one aspect, the sensitivities at about 50% specificity are about 88% for stage I, about 96% for stage II, and about 100% for stages III & IV. In one aspect, the method further includes identifying a subset of proteins from the panel of proteins that contribute to detection benefit. In one aspect, the identification of the subset of proteins is performed using an iterative process that removes the least influential protein in each iteration. In one aspect, the iterative process results in a list of top influential proteins that maximizes performance and lowers the potential cost of the combined assay. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof. In one aspect, the method includes detecting one, two, three, four, five, six, seven, eight, nine, ten, eleven, twelve, thirteen, fourteen, fifteen, twenty, thirty or more proteins in a panel. In one aspect, the one, two, three, four, five, six, seven, eight, nine, ten, eleven, twelve, thirteen, fourteen, fifteen, twenty, thirty or more proteins are selected from ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53. Any combination of proteins ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 may be used in a panel in the methods described herein.

The present disclosure provides a system for detecting lung cancer in an individual. In one aspect, the system includes a. a protein platform configured to measure levels of a panel of literature-curated proteins in a sample from the individual; b. an analyzer configured to analyze cell-free DNA fragmentation patterns in the sample; c. a processor configured to apply a machine learning model to the measured levels of the proteins and the analyzed cell-free DNA fragmentation patterns to determine a combined AUC score; and. d diagnostic module configured to diagnose the presence or stage of lung cancer in the individual based on the combined AUC score. In one aspect, the method includes detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof. In one aspect, the method includes detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof. In one aspect, the method includes detecting one, two, three, four, five, six, seven, eight, nine, ten, eleven, twelve, thirteen, fourteen, fifteen, twenty, thirty or more proteins in a panel. In one aspect, the one, two, three, four, five, six, seven, eight, nine, ten, eleven, twelve, thirteen, fourteen, fifteen, twenty, thirty or more proteins are selected from ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53. Any combination of proteins ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 may be used in a panel in the methods described herein.

The present disclosure also provides a non-transitory computer-readable medium containing instructions that, when executed by a processor, perform a method for detecting lung cancer in an individual. In one aspect, the computer-readable medium includes a. receiving data corresponding to levels of a panel of literature-curated proteins measured in a sample from the individual; b. receiving data corresponding to cell-free DNA fragmentation patterns analyzed in the sample; c. applying a machine learning model to the received data to determine a combined AUC score; and d. outputting a diagnosis for the presence or stage of lung cancer in the individual based on the combined AUC score.

In some aspects, the proteomic component includes a) a multiplexed protein detection assay with an NGS-based readout; b) the ability to multiplex to dozens to tens of thousands proteins or down to a handful of proteins in a single sample; c) the ability to target specific protein content with a cocktail of chosen affinity binding molecules (such as aptamers or antibodies); d) DNA-based barcodes that allow for the association between DNA sequencing results and the source patient and that are compatible with the barcode system in the genomic component; e) a computer analysis pipeline that processes NGS readout of protein assay results associated with each patient. In some aspects, the multiplex protein detection assay is designed to target/analyze from about 10 to about 100,000 proteins in a single sample. In some aspects, the multiplex detection assay is designed to target/analyze from about 10 to about 1000 protein in a single sample. In some aspects, specific proteins can be detected by the proteomic component using a cocktail or combination of affinity binding molecules that recognize the specific proteins. In some aspects, the affinity binding molecules include aptamers or antibodies.

In some aspects, the diagnostic system includes liquid handling robots. Such robots can carry out one or more assay steps of the genomic and proteomic components described herein.

In some aspects, the diagnostic assay system includes a laboratory information management (LIMS) system. In some aspects, the LIMS a) tracks one or more assay steps; b) governs actions of the liquid handling robots; c) tracks which protein content is desired (as indicated by operator selection or a test requisition form) and enforces the use of any protein-content specifying reagent at the appropriate point in the assay d) tracks patient identities of samples and of generated test information (either directly or with a patient-associated code) for both the proteomic and genomic components.

In some aspects, the diagnostic assay system includes a software classifier component that combines information generated by the genomic and proteomic components into a reported risk score for a patient for one or more types of cancer. In some aspects, the software classifier further combines the information related to patient demographic and/or patient health information.

In some aspects, the pooling of NGS libraries from the genomic and proteomic components is performed to allow simultaneous readout of both components.

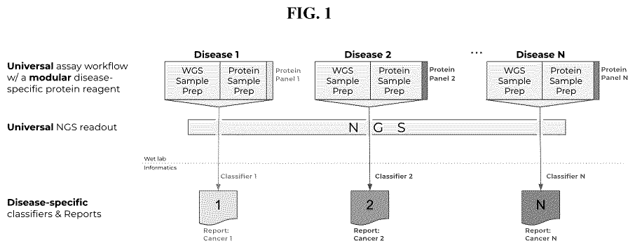

In some aspects, the diagnostic assay system includes a modular protein content design, where two or more disease of interest each have their own associated protein reagents, such that a laboratory that runs multiple tests can run them at the same time on the same robot deck with only the protein reagent, classifier, and report differing among the different disease tests (See FIG. 1).

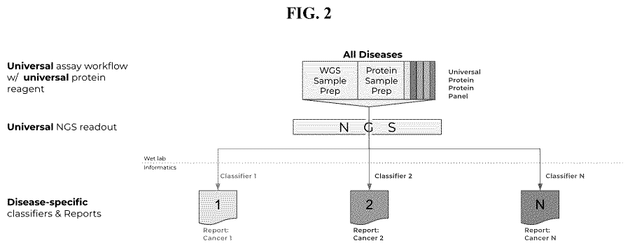

In some aspects, the diagnostic assay system includes a universal protein content design, where a single protein reagent, containing all affinity binding molecules for all tests, is employed; differentiation of employed content for different tests would occur informatically, such as through filtering of sequences associated with certain proteins, followed by use of disease-specific classifiers and reports (See FIG. 2).

In some aspects, the present disclosure provides a proteomic discovery system. The proteomic discovery systems includes the genomic component of the assay system described herein, the proteomic component of the assay system described herein. The proteomic component is utilized using a large discovery panel of protein content. In some aspects, the proteomic discovery system further includes one or more cohorts of patients known to have the disease or diseases in question. In some aspects, the proteomic component of the assay system is run with a large discovery panel of protein content. In some aspects, the proteomic discovery system includes a machine learning algorithm that generates a classifier that combines information generated by the genomic and proteomic components (with or without patient demographic or health information) into a reported risk score for a patient for the disease or diseases in question.

In some aspects, the proteomic component is further configured to allow for the discovery and efficient deployment of integrated genomic and proteomic diagnostic assays, enabling efficient discovery and modular deployment of protein-based panels in the context of a genomic-based workflow.

In some aspects, the present disclosure provides a method for detecting lung cancer in an individual. In some aspects, the method includes a) obtaining a sample from the individual; b) analyzing the sample to detect a presence of a panel of about 100 proteins using a SomaLogic protein platform. In some aspects, about 90 of the proteins are associated with lung cancer risk and about 10 of the proteins are not associated with lung cancer risk; c) assessing cell-free DNA fragmentation patterns in the sample; d) applying a machine learning model to the detected proteins and cell-free DNA fragmentation patterns to generate an area under the curve (AUC) score; and e) determining the presence of lung cancer in the individual based on the AUC score.

In some aspects, the present disclosure provides a system for detecting lung cancer in an individual. The system includes a) SomaLogic protein platform configured to analyze a sample from the individual to detect a presence of a panel of about 100 proteins; b) a cell-free DNA fragmentation analysis module configured to assess cell-free DNA fragmentation patterns in the sample; c) a machine learning module configured to apply a machine learning model to the detected proteins and cell-free DNA fragmentation patterns to generate an AUC score; and d) a diagnostic module configured to determine the presence of lung cancer in the individual based on the AUC score.

In some aspects, the present disclosure provides a non-transitory computer-readable medium storing instructions that, when executed by a processor, causes the processor to perform a method for detecting lung cancer in an individual. In some aspects, the method includes a) receiving data indicative of a presence of a panel of 100 proteins in a sample from the individual, wherein the proteins are analyzed using a SomaLogic protein platform; b) receiving data indicative of cell-free DNA fragmentation patterns in the sample; c) applying a machine learning model to the received data to generate an AUC score; and d) outputting a determination of the presence of lung cancer in the individual based on the AUC score.

In some aspects, the present disclosure provides a method for detecting lung cancer in an individual. In some aspects, the method includes a) measuring levels of a panel of about 100 literature-curated proteins in a sample from the individual using a SomaLogic protein platform; b) analyzing cell-free DNA fragmentation patterns in the sample; c) applying a machine learning model to the measured levels of the 100 proteins and the analyzed cell-free DNA fragmentation patterns to determine a combined area under the curve (AUC) score; and d) diagnosing the presence or stage of lung cancer in the individual based on the combined AUC score. In some aspects, the sample is a L101 sample. In some aspects, the machine learning is a gradient boosting machine learning (GBM) model. In some aspects, a panel of about 100 proteins is associated with lung cancer risk. In some aspects, the machine learning model provides a combined AUC of about 0.86 (0.82-0.9) for the about 100 proteins. In some aspects, the combined AUC for stage I lung cancer is about 0.75 (0.68-0.82) when using the about 100 proteins alone. In some aspects, the combined AUC for detecting lung cancer using both proteins and cell-free DNA fragmentation is about 0.90 (0.87-0.93). In some aspects, the combined AUC for stage I lung cancer using both proteins and cell-free DNA fragmentation is about 0.81 (0.75-0.88). In some aspects, the method, further includes identifying a subset of proteins from the panel of about 100 proteins that contribute to detection benefit, wherein the subset comprises about 20 or fewer proteins. In some aspects, the identification of the subset of proteins is performed using an iterative process that removes the least influential protein in each iteration. In some aspects, the iterative process results in a list of top influential proteins that maximizes performance and lowers the potential cost of the combined assay.

In some aspects, the present disclosure provides a system for detecting lung cancer in an individual. In some aspects, the system includes a) a SomaLogic protein platform configured to measure levels of a panel of about 100 literature-curated proteins in a sample from the individual; b) an analyzer configured to analyze cell-free DNA fragmentation patterns in the sample; c) a processor configured to apply a machine learning model to the measured levels of the about 100 proteins and the analyzed cell-free DNA fragmentation patterns to determine a combined AUC score; and d) a diagnostic module configured to diagnose the presence or stage of lung cancer in the individual based on the combined AUC score.

In some aspects, the present disclosure provides a method for detecting lung cancer in an individual. In some aspects, the method includes a) obtaining a sample from the individual; b) analyzing the sample to detect a presence of a panel of about 100 proteins using a Olink Reveal protein platform. In some aspects, about 90 of the proteins are associated with lung cancer risk and about 10 of the proteins are not associated with lung cancer risk; c) assessing cell-free DNA fragmentation patterns in the sample; d) applying a machine learning model to the detected proteins and cell-free DNA fragmentation patterns to generate an area under the curve (AUC) score; and e) determining the presence of lung cancer in the individual based on the AUC score.

In some aspects, the present disclosure provides a system for detecting lung cancer in an individual. The system includes a) Olink Reveal protein platform configured to analyze a sample from the individual to detect a presence of a panel of about 100 proteins; b) a cell-free DNA fragmentation analysis module configured to assess cell-free DNA fragmentation patterns in the sample; c) a machine learning module configured to apply a machine learning model to the detected proteins and cell-free DNA fragmentation patterns to generate an AUC score; and d) a diagnostic module configured to determine the presence of lung cancer in the individual based on the AUC score.

In some aspects, the present disclosure provides a non-transitory computer-readable medium storing instructions that, when executed by a processor, causes the processor to perform a method for detecting lung cancer in an individual. In some aspects, the method includes a) receiving data indicative of a presence of a panel of 100 proteins in a sample from the individual, wherein the proteins are analyzed using a Olink Reveal protein platform; b) receiving data indicative of cell-free DNA fragmentation patterns in the sample; c) applying a machine learning model to the received data to generate an AUC score; and d) outputting a determination of the presence of lung cancer in the individual based on the AUC score.

In some aspects, the present disclosure provides a method for detecting lung cancer in an individual. In some aspects, the method includes a) measuring levels of a panel of about 100 literature-curated proteins in a sample from the individual using a Olink Reveal protein platform; b) analyzing cell-free DNA fragmentation patterns in the sample; c) applying a machine learning model to the measured levels of the 100 proteins and the analyzed cell-free DNA fragmentation patterns to determine a combined area under the curve (AUC) score; and d) diagnosing the presence or stage of lung cancer in the individual based on the combined AUC score. In some aspects, the sample is a L101 sample. In some aspects, the machine learning is a gradient boosting machine learning (GBM) model. In some aspects, a panel of about 100 proteins is associated with lung cancer risk. In some aspects, the machine learning model provides a combined AUC of about 0.86 (0.82-0.9) for the about 100 proteins. In some aspects, the combined AUC for stage I lung cancer is about 0.75 (0.68-0.82) when using the about 100 proteins alone. In some aspects, the combined AUC for detecting lung cancer using both proteins and cell-free DNA fragmentation is about 0.90 (0.87-0.93). In some aspects, the combined AUC for stage I lung cancer using both proteins and cell-free DNA fragmentation is about 0.81 (0.75-0.88). In some aspects, the method, further includes identifying a subset of proteins from the panel of about 100 proteins that contribute to detection benefit, wherein the subset comprises about 20 or fewer proteins. In some aspects, the identification of the subset of proteins is performed using an iterative process that removes the least influential protein in each iteration. In some aspects, the iterative process results in a list of top influential proteins that maximizes performance and lowers the potential cost of the combined assay.

In some aspects, the present disclosure provides a system for detecting lung cancer in an individual. In some aspects, the system includes a) a Olink Reveal protein platform configured to measure levels of a panel of about 100 literature-curated proteins in a sample from the individual; b) an analyzer configured to analyze cell-free DNA fragmentation patterns in the sample; c) a processor configured to apply a machine learning model to the measured levels of the about 100 proteins and the analyzed cell-free DNA fragmentation patterns to determine a combined AUC score; and d) a diagnostic module configured to diagnose the presence or stage of lung cancer in the individual based on the combined AUC score.

In some aspects, the present disclosure provides a method for detecting lung cancer in an individual. In some aspects, the method includes a) obtaining a sample from the individual; b) analyzing the sample to detect a presence of a panel of about 86 proteins as depicted in Table 4; c) assessing cell-free DNA fragmentation patterns in the sample; d) applying a machine learning model to the detected proteins and cell-free DNA fragmentation patterns to generate an area under the curve (AUC) score; and e) determining the presence of lung cancer in the individual based on the AUC score.

In some aspects, the present disclosure provides a system for detecting lung cancer in an individual. The system includes a) a protein detection platform configured to analyze a sample from the individual to detect a presence of a panel of about 86 proteins as depicted in Table 4; b) a cell-free DNA fragmentation analysis module configured to assess cell-free DNA fragmentation patterns in the sample; c) a machine learning module configured to apply a machine learning model to the detected proteins and cell-free DNA fragmentation patterns to generate an AUC score; and d) a diagnostic module configured to determine the presence of lung cancer in the individual based on the AUC score.

In some aspects, the present disclosure provides a non-transitory computer-readable medium storing instructions that, when executed by a processor, causes the processor to perform a method for detecting lung cancer in an individual. In some aspects, the method includes a) receiving data indicative of a panel of about 86 proteins as depicted in Table 4 in a sample from the individual, wherein the proteins are analyzed using a SomaLogic protein platform; b) receiving data indicative of cell-free DNA fragmentation patterns in the sample; c) applying a machine learning model to the received data to generate an AUC score; and d) outputting a determination of the presence of lung cancer in the individual based on the AUC score.

In some aspects, the present disclosure provides a method for detecting lung cancer in an individual. In some aspects, the method includes a) measuring levels of a panel of about 86 proteins as depicted in Table 4 in a sample from the individual using a protein platform; b) analyzing cell-free DNA fragmentation patterns in the sample; c) applying a machine learning model to the measured levels of the a about 86 proteins depicted in Table 4 and the analyzed cell-free DNA fragmentation patterns to determine a combined area under the curve (AUC) score; and d) diagnosing the presence or stage of lung cancer in the individual based on the combined AUC score. In some aspects, the sample is a L101 sample. In some aspects, the machine learning is a gradient boosting machine learning (GBM) model. In some aspects, a panel of about 86 proteins as depicted in Table 4 is associated with lung cancer risk. In some aspects, the machine learning model provides a combined AUC of about 0.86 (0.82-0.9) for the panel of about 86 proteins as depicted in Table 4. In some aspects, the combined AUC for stage I lung cancer is about 0.75 (0.68-0.82) when using the panel of about 86 proteins as depicted in Table 4 alone. In some aspects, the combined AUC for detecting lung cancer using both proteins and cell-free DNA fragmentation is about 0.90 (0.87-0.93). In some aspects, the combined AUC for stage I lung cancer using both proteins and cell-free DNA fragmentation is about 0.81 (0.75-0.88). In some aspects, the method, further includes identifying a subset of proteins from the panel of about 86 proteins as depicted in Table 4 that contribute to detection benefit, wherein the subset comprises about 20 or fewer proteins. In some aspects, the identification of the subset of proteins is performed using an iterative process that removes the least influential protein in each iteration. In some aspects, the iterative process results in a list of top influential proteins that maximizes performance and lowers the potential cost of the combined assay.

In some aspects, the present disclosure provides a system for detecting lung cancer in an individual. In some aspects, the system includes a) a protein detection platform configured to measure levels of a panel of about 86 proteins as depicted in Table 4 in a sample from the individual; b) an analyzer configured to analyze cell-free DNA fragmentation patterns in the sample; c) a processor configured to apply a machine learning model to the measured levels of the panel of about 86 proteins as depicted in Table 4 and the analyzed cell-free DNA fragmentation patterns to determine a combined AUC score; and d) a diagnostic module configured to diagnose the presence or stage of lung cancer in the individual based on the combined AUC score.

In some aspects, the present disclosure provides a method for detecting lung cancer in an individual. In some aspects, the method includes a) obtaining a sample from the individual; b) analyzing the sample to detect a presence of a panel of about 47 proteins as depicted in Table 5; c) assessing cell-free DNA fragmentation patterns in the sample; d) applying a machine learning model to the detected proteins and cell-free DNA fragmentation patterns to generate an area under the curve (AUC) score; and e) determining the presence of lung cancer in the individual based on the AUC score.

In some aspects, the present disclosure provides a system for detecting lung cancer in an individual. The system includes a) a protein detection platform configured to analyze a sample from the individual to detect a presence of a panel of about 47 proteins as depicted in Table 5; b) a cell-free DNA fragmentation analysis module configured to assess cell-free DNA fragmentation patterns in the sample; c) a machine learning module configured to apply a machine learning model to the detected proteins and cell-free DNA fragmentation patterns to generate an AUC score; and d) a diagnostic module configured to determine the presence of lung cancer in the individual based on the AUC score.

In some aspects, the present disclosure provides a non-transitory computer-readable medium storing instructions that, when executed by a processor, causes the processor to perform a method for detecting lung cancer in an individual. In some aspects, the method includes a) receiving data indicative of a panel of about 47 proteins as depicted in Table 5 in a sample from the individual, wherein the proteins are analyzed using a SomaLogic protein platform; b) receiving data indicative of cell-free DNA fragmentation patterns in the sample; c) applying a machine learning model to the received data to generate an AUC score; and d) outputting a determination of the presence of lung cancer in the individual based on the AUC score.

In some aspects, the present disclosure provides a method for detecting lung cancer in an individual. In some aspects, the method includes a) measuring levels of a panel of about 47 proteins as depicted in Table 5 in a sample from the individual using a protein platform; b) analyzing cell-free DNA fragmentation patterns in the sample; c) applying a machine learning model to the measured levels of the a about 47 proteins depicted in Table 5 and the analyzed cell-free DNA fragmentation patterns to determine a combined area under the curve (AUC) score; and d) diagnosing the presence or stage of lung cancer in the individual based on the combined AUC score. In some aspects, the sample is a L101 sample. In some aspects, the machine learning is a gradient boosting machine learning (GBM) model. In some aspects, a panel of about 47 proteins as depicted in Table 5 is associated with lung cancer risk. In some aspects, the machine learning model provides a combined AUC of about 0.86 (0.82-0.9) for the panel of about 47 proteins as depicted in Table 5. In some aspects, the combined AUC for stage I lung cancer is about 0.75 (0.68-0.82) when using the panel of about 47 proteins as depicted in Table 5 alone. In some aspects, the combined AUC for detecting lung cancer using both proteins and cell-free DNA fragmentation is about 0.90 (0.87-0.93). In some aspects, the combined AUC for stage I lung cancer using both proteins and cell-free DNA fragmentation is about 0.81 (0.75-0.88). In some aspects, the method further includes identifying a subset of proteins from the panel of about 47 proteins as depicted in Table 5 that contribute to detection benefit, wherein the subset comprises about 20 or fewer proteins. In some aspects, the identification of the subset of proteins is performed using an iterative process that removes the least influential protein in each iteration. In some aspects, the iterative process results in a list of top influential proteins that maximizes performance and lowers the potential cost of the combined assay.

In some aspects, the present disclosure provides a system for detecting lung cancer in an individual. In some aspects, the system includes a) a protein detection platform configured to measure levels of a panel of about 47 proteins as depicted in Table 5 in a sample from the individual; b) an analyzer configured to analyze cell-free DNA fragmentation patterns in the sample; c) a processor configured to apply a machine learning model to the measured levels of the panel of about 47 proteins as depicted in Table 5 and the analyzed cell-free DNA fragmentation patterns to determine a combined AUC score; and d) a diagnostic module configured to diagnose the presence or stage of lung cancer in the individual based on the combined AUC score.

In some aspects, the present disclosure provides a non-transitory computer-readable medium containing instructions that, when executed by a processor, perform a method for detecting lung cancer in an individual. In some aspects, the method includes a) receiving data corresponding to levels of a panel of about 100 literature-curated proteins measured in a sample from the individual; b) receiving data corresponding to cell-free DNA fragmentation patterns analyzed in the sample; c) applying a machine learning model to the received data to determine a combined AUC score; and d) outputting a diagnosis for the presence or stage of lung cancer in the individual based on the combined AUC score.

In some aspects, the sample is a L101 sample. In some aspects, the L101 sample are obtained from a study designed to train and test classifiers for lung cancer detection using the DELFI assay and other biomarker and clinical features. In some aspects, L101 sample is obtained from DELFI's prospective, observational case-control study to train and validate classifier for LDT lung cancer detection and multi-cancer detection as described in clinical trial with clinicaltrials.gov ID: NCT04825834 (https://www.cancer.gov/research/participate/clinical-trials-search/v?id=NCI-2022-02585).

In some aspects, the machine learning model includes a gradient boosting machine (GBM) model. In some aspects, the AUC score for the combined analysis of proteins and cell-free DNA fragmentation patterns is at least 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99 or more. In some aspects, the AUC score for stage I lung cancer is at least about 0.81. In some aspects, the methods disclosed herein include evaluating the performance of the combined protein and cell-free DNA fragmentation model at 50% specificity; and/or determining the sensitivity for detecting stage I, stage II, and stage III & IV lung cancer. In some aspects, the sensitivity for detecting stage I lung cancer is at least about 85, %, 86%, 87%, 88%, 89%, 90%, 91%, 92%, 93%, 94%, 95%, 96%, 97%, 98%, 99% or more. As a non-limiting example, the sensitivity for detecting stage I lung cancer is at least 88%.

In some aspects, the sensitivity for detecting stage II lung cancer is at least about 85, %, 86%, 87%, 88%, 89%, 90%, 91%, 92%, 93%, 94%, 95%, 96%, 97%, 98%, 99% or more. As a non-limiting example, the sensitivity for detecting stage I lung cancer is at least 96%.

In some aspects, the sensitivity for detecting stage III and/or stage IV lung cancer is at least about 85, 0% 86%, 87%, 88%, 89%, 90%, 91%, 92%, 93%, 94%, 95%, 96%, 97%, 98%, 99% or more. As a non-limiting example, the sensitivity for detecting stage III and/or IV lung cancer is about 100%.

In some aspects, a cfDNA fragmentation profile includes a cfDNA fragment size pattern. cfDNA fragments can be any appropriate size. In some aspects, the cfDNA fragment is from about 50 base pairs (bp) to about 400 bp in length. In some aspects, a mammal having cancer has a cfDNA fragment size pattern that contains a shorter median cfDNA fragment size than the median cfDNA fragment size in a healthy mammal. In some aspects, a healthy mammal (e.g., a mammal not having cancer) has cfDNA fragment sizes having a median cfDNA fragment size from about 166.6 bp to about 167.2 bp (e.g., about 166.9 bp). In some aspects, a mammal having cancer has cfDNA fragment sizes that are, on average, about 1.28 bp to about 2.49 bp (e.g., about 1.88 bp) shorter than cfDNA fragment sizes in a healthy mammal. In some aspects, a mammal having cancer has cfDNA fragment sizes having a median cfDNA fragment size of about 164.11 bp to about 165.92 bp (e.g., about 165.02 bp).

In some aspects, the cfDNA fragmentation profile includes a cfDNA fragment size distribution. In some aspects, a mammal having cancer has a cfDNA size distribution that is more variable than a cfDNA fragment size distribution in a healthy mammal. In some aspects, a size distribution is within a targeted region. In some aspects, a healthy mammal (e.g., a mammal not having cancer) has a targeted region cfDNA fragment size distribution of about 1 or less than about 1. In some aspects, a mammal having cancer has a targeted region cfDNA fragment size distribution that is longer (e.g., 10, 15, 20, 25, 30, 35, 40, 45, 50 or more bp longer, or any number of base pairs between these numbers) than a targeted region cfDNA fragment size distribution in a healthy mammal. In some aspects, a mammal having cancer has a targeted region cfDNA fragment size distribution that is shorter (e.g., 10, 15, 20, 25, 30, 35, 40, 45, 50 or more bp shorter, or any number of base pairs between these numbers) than a targeted region cfDNA fragment size distribution in a healthy mammal. In some aspects, a mammal having cancer has a targeted region cfDNA fragment size distribution that is about 47 bp smaller to about 30 bp longer than a targeted region cfDNA fragment size distribution in a healthy mammal. In some aspects, a mammal having cancer can have a targeted region cfDNA fragment size distribution of, on average, a 10, 11, 12, 13, 14, 15, 15, 17, 18, 19, 20 or more bp difference in lengths of cfDNA fragments. For example, a mammal having cancer has a targeted region cfDNA fragment size distribution of, on average, about a 13 bp difference in lengths of cfDNA fragments. In some aspects, a size distribution is a genome-wide size distribution. In some aspects, a healthy mammal (e.g., a mammal not having cancer) has a very similar distributions of short and long cfDNA fragments genome-wide. In some aspects, a mammal having cancer has, genome-wide, one or more alterations (e.g., increases and decreases) in cfDNA fragment sizes. In some aspects, the one or more alterations is any appropriate chromosomal region of the genome. For example, an alteration is in a portion of a chromosome. Examples of chromosomes that contain one or more alterations in cfDNA fragment sizes include, without limitation, portions of 2 q, 4 p, 5 p, 6 q, 7 p, 8 q, 9 q, 10 q, 11 q, 12 q, and 14 q. For example, an alteration is across a chromosome arm (e.g., an entire chromosome arm).

In some aspects, a cfDNA fragmentation profile includes a ratio of small cfDNA fragments to large cfDNA fragments and a correlation of fragment ratios to reference fragment ratios. As used herein, with respect to ratios of small cfDNA fragments to large cfDNA fragments, a small cfDNA fragment is from about 100 bp in length to about 150 bp in length. As used herein, with respect to ratios of small cfDNA fragments to large cfDNA fragments, a large cfDNA fragment is from about 151 bp in length to 220 bp in length. As described herein, a mammal having cancer has a correlation of fragment ratios (e.g., a correlation of cfDNA fragment ratios to reference DNA fragment ratios such as DNA fragment ratios from one or more healthy mammals) that is lower (e.g., 2-fold lower, 3-fold lower, 4-fold lower, 5-fold lower, 6-fold lower, 7-fold lower, 8-fold lower, 9-fold lower, 10-fold lower, or more) than in a healthy mammal. In some aspects, a healthy mammal (e.g., a mammal not having cancer) has a correlation of fragment ratios (e.g., a correlation of cfDNA fragment ratios to reference DNA fragment ratios such as DNA fragment ratios from one or more healthy mammals) of about 1 (e.g., about 0.96). In some aspects, a mammal having cancer has a correlation of fragment ratios (e.g., a correlation of cfDNA fragment ratios to reference DNA fragment ratios such as DNA fragment ratios from one or more healthy mammals) that is, on average, about 0.19 to about 0.30 (e.g., about 0.25) lower than a correlation of fragment ratios (e.g., a correlation of cfDNA fragment ratios to reference DNA fragment ratios such as DNA fragment ratios from one or more healthy mammals) in a healthy mammal.

In some aspects, a cfDNA fragmentation profile includes coverage of all fragments. Coverage of all fragments can include windows (e.g., non-overlapping windows) of coverage. In some aspects, coverage of all fragments includes windows of small fragments (e.g., fragments from about 100 bp to about 150 bp in length). In some aspects, coverage of all fragments includes windows of large fragments (e.g., fragments from about 151 bp to about 220 bp in length).

In some aspects, a cfDNA fragmentation profile is obtained using any appropriate method. In some aspects, cfDNA from a mammal (e.g., a mammal having, or suspected of having, cancer) is processed into sequencing libraries which are subjected to whole genome sequencing (e.g., low-coverage whole genome sequencing), mapped to the genome, and analyzed to determine cfDNA fragment lengths. Mapped sequences are analyzed in non-overlapping windows covering the genome. In some aspects, windows can be any appropriate size. For example, windows are from thousands to millions of bases in length. As one non-limiting example, a window is about 5 megabases (Mb) long. In some aspects, any appropriate number of windows are mapped. For example, tens to thousands of windows are mapped in the genome. For example, hundreds to thousands of windows are mapped in the genome. In some aspects, a cfDNA fragmentation profile is determined within each window. In some aspects, the low-coverage whole genome sequencing can include sequencing at a depth of less than 10× genome coverage. In some aspects, the low-coverage genome sequencing can include sequencing at a depth of about 0.1× to 10× genome coverage. In some aspects, the low-coverage genome sequencing can include sequencing at a depth of about 9×, 8×, 7×, 6×, 5×, 4×, 3×, 2×, 1×, 0.5×, 0.4×, 0.3×, 0.2×, 0.1× or less genome coverage.

In some aspects, the proteomic component includes a protein detection assay. In some aspects, the protein detection assay is high multiplex affinity proteomics profiling assay such as but not limited to an Olink Bioscience assay (Uppsala, Sweden) or a SOMAscan assay (SomaLogic: Boulder, CO). The Olink Bioscience proteomics platform provides multiplexed immune-based assay panels targeted toward various disease processes. The SOMAscan platform and SomaLogic provides modified oligonucleotide aptamer-based assays that cover a broad range of biological processes.

Any appropriate sample from a mammal is assessed as described herein (e.g., assessed for a DNA fragmentation pattern or proteomic components). In some aspects, a sample includes DNA (e.g., genomic DNA) or protein. In some aspects, a sample includes cfDNA (e.g., circulating tumor DNA (ctDNA)). In some aspects, a sample is a fluid sample (e.g., a liquid biopsy). Examples of samples that contain DNA and/or polypeptides include, without limitation, blood (e.g., whole blood, serum, or plasma), amnion, tissue, urine, cerebrospinal fluid, saliva, sputum, broncho-alveolar lavage, bile, lymphatic fluid, cyst fluid, stool, ascites, pap smears, breast milk, and exhaled breath condensate. For example, a plasma sample can be assessed to determine a cfDNA fragmentation profile and proteomic component.

In some aspects, a sample is processed (e.g., to isolate and/or purify DNA and/or polypeptides from the sample). For example, DNA isolation and/or purification includes cell lysis (e.g., using detergents and/or surfactants), protein removal (e.g., using a protease), and/or RNA removal (e.g., using an RNase). As another example, polypeptide isolation and/or purification includes cell lysis (e.g., using detergents and/or surfactants), DNA removal (e.g., using a DNase), and/or RNA removal (e.g., using an RNase).

In some aspects, a mammal having, or suspected of having, any appropriate type of cancer is assessed (e.g., to determine a cfDNA fragmentation profile) and/or treated (e.g., by administering one or more cancer treatments to the mammal) using the methods and materials described herein. A cancer can be any stage cancer. In some aspects, a cancer is an early stage cancer. In some cases, a cancer is an asymptomatic cancer. In some aspects, a cancer is a residual disease and/or a recurrence (e.g., after surgical resection and/or after cancer therapy). In some aspects, a cancer is any type of cancer. Examples of types of cancers that are assessed, monitored, and/or treated as described herein include, without limitation, lung cancer, colorectal cancers, breast cancers, gastric cancers, pancreatic cancers, bile duct cancers, and ovarian cancers.

In some aspects, the cancer is stage I, stage II, stage III or stage IV cancer.

When treating a mammal having, or suspected of having, cancer as described herein, the mammal is administered one or more cancer treatments. In some aspects, a cancer treatment can be any appropriate cancer treatment. One or more cancer treatments described herein are administered to a mammal at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g. a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the mammal.

In some aspects, a cancer treatment can include an immune checkpoint inhibitor. Non-limiting examples of immune checkpoint inhibitors include nivolumab (Opdivo), pembrolizumab (Keytruda), atezolizumab (tecentriq), avelumab (bavencio), durvalumab (imfinzi), ipilimumab (yervoy). See, e.g., Pardoll (2012) Nat. Rev. Cancer 12: 252-264; Sun et al. (2017) Eur. Rev. Med. Pharmacol. Sci. 21(6): 1198-1205; Hamanishi et al. (2015) J. Clin. Oncol. 33(34): 4015-22; Brahmer et al. (2012) N. Engl. J. Med. 366(26): 2455-65; Ricciuti et al. (2017) J. Thorac. Oncol. 12(5): e51-e55; Ellis et al. (2017) Clin. Lung Cancer pii: 51525-7304(17)30043-8; Zou and Awad (2017) Ann. Oncol. 28(4): 685-687; Sorscher (2017) N. Engl. J. Med. 376(10: 996-7; Hui et al. (2017) Ann. Oncol. 28(4): 874-881; Vansteenkiste et al. (2017) Expert Opin. Biol. Ther. 17(6): 781-789; Hellmann et al. (2017) Lancet Oncol. 18(1): 31-41, Chen (2017) J. Chin. Med. Assoc. 80(1): 7-14.

In some aspects, a cancer treatment is an adoptive T cell therapy (e.g., chimeric antigen receptors and/or T cells having wild-type or modified T cell receptors). See, e.g., Rosenberg and Restifo (2015) Science 348(6230): 62-68; Chang and Chen (2017) Trends Mol. Med. 23(5): 430-450; Yee and Lizee (2016) Cancer J. 23(2): 144-148; Chen et al. (2016) Oncoimmunology 6(2): e1273302; US 2016/0194404; US 2014/0050788; US 2014/0271635; U.S. Pat. No. 9,233,125; incorporated by reference in their entirety herein.

In some aspects, a cancer treatment is a chemotherapeutic agent. Non-limiting examples of chemotherapeutic agents include: amsacrine, azacitidine, axathioprine, bevacizumab (or an antigen-binding fragment thereof), bleomycin, busulfan, carboplatin, capecitabine, chlorambucil, cisplatin, cyclophosphamide, cytarabine, dacarbazine, daunorubicin, docetaxel, doxifluridine, doxorubicin, epirubicin, erlotinib hydrochlorides, etoposide, fiudarabine, floxuridine, fludarabine, fluorouracil, gemcitabine, hydroxyurea, idarubicin, ifosfamide, irinotecan, lomustine, mechlorethamine, melphalan, mercaptopurine, methotrxate, mitomycin, mitoxantrone, oxaliplatin, paclitaxel, pemetrexed, procarbazine, all-trans retinoic acid, streptozocin, tafluposide, temozolomide, teniposide, tioguanine, topotecan, uramustine, valrubicin, vinblastine, vincristine, vindesine, vinorelbine, and combinations thereof. Additional examples of anti-cancer therapies are known in the art; see, e.g. the guidelines for therapy from the American Society of Clinical Oncology (ASCO), European Society for Medical Oncology (ESMO), or National Comprehensive Cancer Network (NCCN).

### EXAMPLES

### Example 1: Workflows for Discovery and Deployment of Diagnostic Assays Combining Proteomic and Genomic Information

FIG. 1 depicts an assay system with modular protein reagent design.

FIG. 2 depicts an assay system with universal protein reagent design.

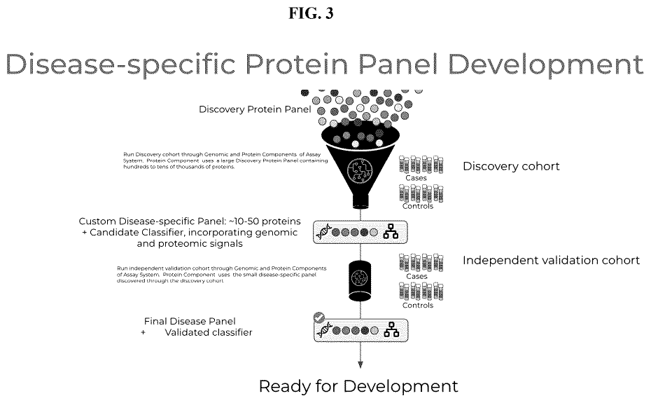

FIG. 3 depicts the development of a disease-specific panel for use in a modular protein reagent design. The discovery of a universal protein reagent could occur by the same approach, either by using discovery cohorts covering many different diseases, or by simply pooling the content of many modular protein reagents for different diseases that had been discovered by multiple such efforts over time; this latter approach would allow a laboratory to start out taking the modular approach and then later decide to switch to a universal approach once a critical mass of modular panels had been developed.

### Example 2: Detection of Lung Cancer Using Proteins and Cell-Free DNA Fragmentomes

The disclosed invention also describes the use of 100 literature-curated proteins alone and or in combination with cell-free DNA fragmentation patterns assessed by machine learning to detect cancer. Briefly, 100 proteins (most of which were associated in the literature with lung cancer risk) were evaluated using the SomaLogic protein platform (See Table 1). We assessed these 100 proteins in 511 L101 samples containing non-cancers and samples ranging from stage I to IV. Initial area under the curve (AUCs) of the top 5 proteins alone resulted in AUCs of 0.69 (0.63-0.74) to 0.76 (0.71-0.81). Including the 100 proteins in a machine learning model led to a combined AUC of 0.86 (0.82-0.9), and for stage I an AUC of 0.75 (0.68-0.82). Lastly, combining proteins and cell-free DNA fragmentation led to an overall AUC of 0.90 (0.87-0.93) or stage I AUC of 0.81 (0.75-0.88). If we evaluate the performance of the combined protein and cell-free DNA fragmentation model at 50% specificity this resulted in sensitivities of stage I (88%), stage II (96%), stage III & IV (100%). Next steps will be to validate on more samples as well as externally validate this approach with the potential aim to lower the protein number for 100 proteins to key top proteins in a way that maximizes performance and lowers potential cost of the combined assay.

For high-risk lung cancer screening, current approaches using cell-free DNA fragmentomes have been promising (Mathios et al. 2020). Here we evaluate cell-free DNA fragmentomes and use the matched plasma for protein analysis. The incorporation of a specific set of proteins curated from the literature of high-risk lung cancer individuals together with cell-free DNA fragmentomes provides better performance than either feature alone.

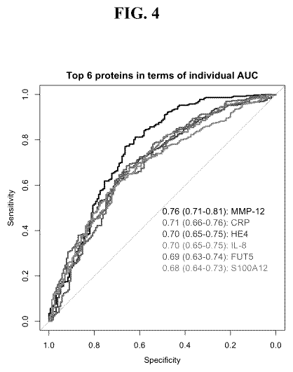

FIG. 4 depicts the performance of the top 6 individual proteins (See also Table 2).

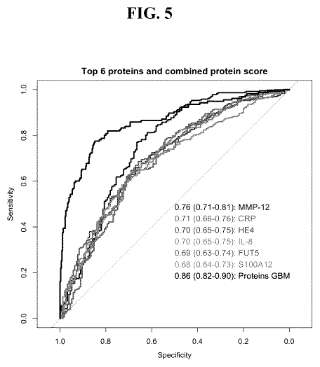

FIG. 5 depicts the performance of a combined protein GBM model.

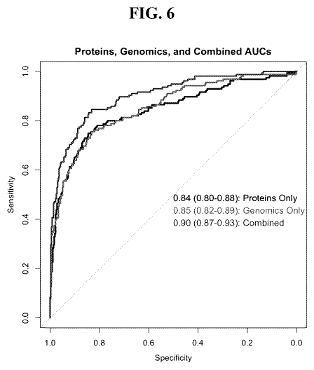

FIG. 6 depicts the performance of a combined protein and cell-free DNA fragmentation approach.

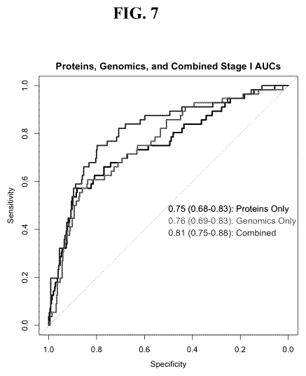

FIG. 7 depicts the performance of a combined protein and cell-free DNA fragmentation approach in stage I lung cancer individuals.

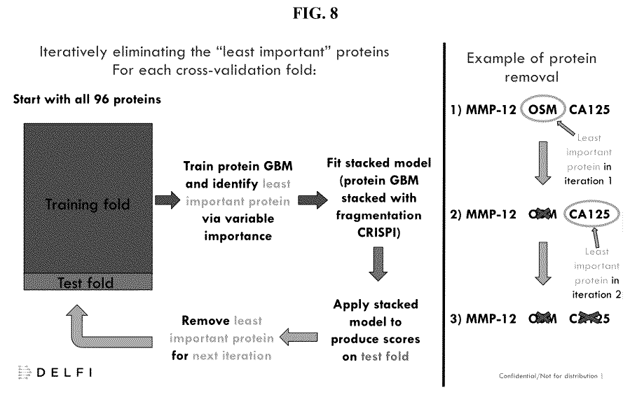

FIG. 8 depicts the approach for identifying proteins that greatly contribute detection benefit to potentially reduce 100 proteins to a list of 20 or less, for potential reduction in cost of an assay including both fragmentation and proteins.

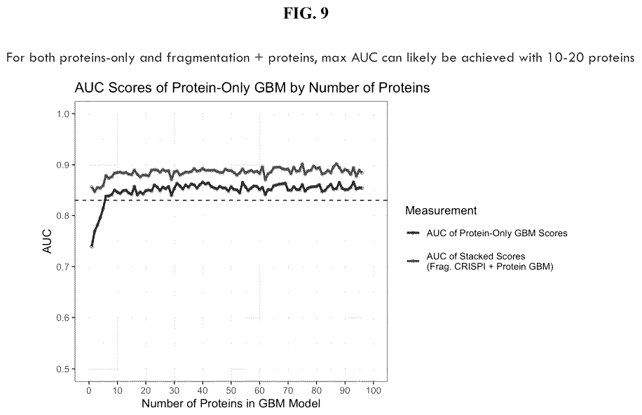

FIG. 9 depicts the model performance following schema in FIG. 8, where the least influential protein is removed in each iteration.

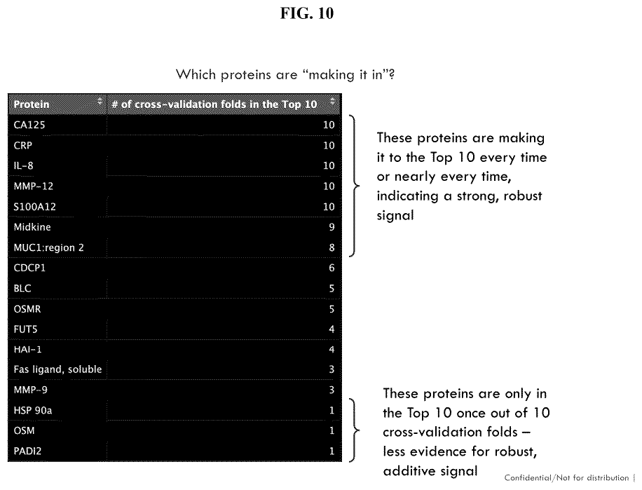

FIG. 10 depicts a list of top influential proteins after following the schema in FIG. 8 (See also Table 3).

TABLE 1
Evaluation of lung cancer risk associated proteins using the SomaLogic protein platform

| List of 100 | # in SL | Custom | SOMAmer | | | |
|---|---|---|---|---|---|---|
| proteins | panel | Panel (X) | SeqID | Target Name | Human Target or Analyte | UniProt ID |
| 1 | 126 | X | 3175-51 | ATS13 | A disintegrin and | Q76LX8 |
| | | | | | metalloproteinase with | |
| | | | | | thrombospondin motifs 13 | |
| 2 | 192 | X | 21440-9 | ADAM-8 | ADAM 8 | P78325 |
| 3 | 289 | X | 11480-1 | Aldehyde dehydrogenase, | Aldehydede hydrogenase, | P30838 |
| | | | | class 3 | dimeric NADP-preferring | |
| 4 | 300 | X | 7813-6 | Alkaline phosphatase, | Alkaline phosphatase, | P05187 |
| | | | | placental | placental type | |
| 5 | 311 | X | 4549-78 | FUT5 | Alpha-(1,3)-fucosyltrans- | Q11128 |
| | | | | | ferase 5 | |
| 6 | 325 | X | 3580-25 | a1-Antitrypsin | Alpha-1-antitrypsin | P01009 |
| 7 | 348 | X | 5792-8 | AFP | alpha-Fetoprotein | P02771 |
| 8 | 384 | X | 2970-60 | AREG | Amphiregulin | P15514 |
| 9 | 413 | X | 2602-2 | Angiopoietin-2 | Angiopoietin-2 | O15123 |
| 10 | 444 | X | 4960-72 | annexin 1 | Annexin A1 | P04083 |
| 11 | 666 | X | 7203-125 | RFNG | Beta-1,3-N-acetylglucos- | Q9Y644 |
| | | | | | aminyltransferase radical fringe | |
| 12 | 894 | X | 15545-13 | Calcineurin B a | Calcineurin subunit B type 1 | P63098 |
| 13 | 1071 | X | 18158-45 | Caspase-8 | Caspase-8 | Q14790 |
| 14 | 1093 | X | 3364-76 | Cathepsin V | Cathepsin L2 | O60911 |
| 15 | 1130 | X | 22969-12 | MCP-3 | C-C motif chemokine 7 | P80098 |
| 16 | 1198 | X | 6123-69 | p53 | Cellular tumor antigen p53 | P04637 |
| 17 | 1237 | X | 11104-13 | YKL-40 | Chitinase-3-like protein 1 | P36222 |
| 18 | 1264 | X | 21391-17 | b-CF | Choriogonadotropin | P0DN86 |
| | | | | | subunit beta 3 | |
| 19 | 1315 | X | 8287-17 | CLM2 | CMRF35-like molecule 2 | Q496F6 |
| 20 | 1461 | X | 3666-17 | complement factor | Complement factor | Q9BXR6 |
| | | | | H-related 5 | H-related protein 5 | |
| 21 | 1504 | X | 4337-49 | CRP | C-reactive protein | P02741 |
| 22 | 1528 | X | 7752-31 | CLC4D | C-type lectin domain | Q8WXI8 |
| | | | | | family 4 member D | |
| 23 | 1542 | X | 6565-68 | CDCP1 | CUB domain-containing | Q9H5V8 |
| | | | | | protein 1 | |
| 24 | 1559 | X | 13701-2 | BLC | C-X-C motif chemokine 13 | O43927 |
| 25 | 1563 | X | 9495-10 | VCC1 | C-X-C motif chemokine 17 | Q6UXB2 |
| 26 | 1567 | X | 9188-119 | MIG | C-X-C motif chemokine 9 | Q07325 |
| 27 | 1605 | X | 19768-13 | Cystatin B | Cystatin B | P04080 |
| 28 | 2332 | X | 15583-18 | FCRLB | Fc receptor-like B | Q6BAA4 |
| 29 | 2338 | X | 6103-70 | FCRL5 | Fc receptor-like protein 5 | Q96RD9 |
| 30 | 2364 | X | 3025-50 | bFGF | Fibroblast growth factor 2 | P09038 |
| 31 | 2367 | X | 3807-1 | FGF23 | Fibroblast growth factor 23 | Q9GZV9 |
| 32 | 2499 | X | 4548-4 | Fucosyltrans-ferase 3 | Galactoside 3(4)-L-fucosyl- | P21217 |
| | | | | | transferase | |
| 33 | 2510 | X | 5000-52 | LG3BP | Galectin-3-binding protein | Q08380 |
| 34 | 2529 | X | 11083-23 | NSE | Gamma-enolase | P09104 |
| 35 | 2545 | X | 5897-58 | Gastrin-releasing | Gastrin-releasing peptide | P07492 |
| | | | | peptide | | |
| 36 | 2560 | X | 4775-34 | Gelsolin | Gelsolin | P06396 |
| 37 | 2655 | X | 8867-18 | Glycodelin | Glycodelin | P09466 |
| 38 | 2748 | X | 4374-45 | MIC-1 | Growth/differentiation | Q99988 |
| | | | | | factor 15 | |
| 39 | 2881 | X | 2681-23 | HGF | Hepatocyte growth factor | P14210 |
| 40 | 3029 | X | 2625-53 | HSP 90a | Hsp90alpha | P07900 |
| 41 | 3067 | X | 4987-17 | FCAR | Immunoglobulin alpha Fc | P24071 |
| | | | | | receptor | |
| 42 | 3086 | X | 19587-12 | IMA5 | Importin subunit alpha-5 | P52294 |
| 43 | 3090 | X | 7890-68 | DPP10 | Inactive dipeptidyl peptidase 10 | Q8N608 |
| 44 | 3145 | X | 13741-36 | IGFBP-1 | Insulin-like growth factor- | P08833 |
| | | | | | binding protein 1 | |
| 45 | 3148 | X | 2570-72 | IGFBP-2 | Insulin-like growth factor- | P18065 |
| | | | | | binding protein 2 | |
| 46 | 3204 | X | 4342-10 | sICAM-1 | Intercellular adhesion | P05362 |
| | | | | | molecule 1 | |
| 47 | 3231 | X | 15346-31 | IFN-g | Interferon gamma | P01579 |
| 48 | 3277 | X | 10344-334 | IL-10 Ra | Interleukin-10 receptor | Q13651 |
| | | | | | subunit alpha | |
| 49 | 3318 | X | 3151-6 | IL-2 sRa | Interleukin-2 receptor | P01589 |
| | | | | | subunit alpha | |
| 50 | 3359 | X | 4673-13 | IL-6 | Interleukin-6 | P05231 |
| 51 | 3367 | X | 3447-64 | IL-8 | Interleukin-8 | P10145 |
| 52 | 3453 | X | 15606-19 | Keratin 19 | Keratin, type I cytoskeletal 19 | P08727 |
| 53 | 3509 | X | 9377-25 | SCF | Kit ligand | P21583 |
| 54 | 3517 | X | 2828-82 | HAI-1 | Kunitz-type protease inhibitor 1 | O43278 |
| 55 | 3568 | X | 8484-24 | Leptin | Leptin | P41159 |
| 56 | 3675 | X | 21987-76 | LPL | Lipoprotein lipase | P06858 |
| 57 | 3727 | X | 13107-9 | LYPD3 | Ly6/PLAUR domain-containing | O95274 |
| | | | | | protein 3 | |
| 58 | 3776 | X | 4496-60 | MMP-12 | Macrophage metalloelastase | P39900 |
| 59 | 3840 | X | 2789-26 | MMP-7 | Matrilysin | P09237 |
| 60 | 3851 | X | 2579-17 | MMP-9 | Matrix metalloproteinase-9 | P14780 |
| 61 | 3877 | X | 20075-130 | MAGE-4 | Melanoma-associated antigen 4 | P43358 |
| 62 | 3905 | X | 3893-64 | Mesothelin | Mesothelin | Q13421 |
| 63 | 3910 | X | 23173-3 | TIMP-1 | Metalloproteinase inhibitor 1 | P01033 |
| 64 | 3965 | X | 2911-27 | Midkine | Midkine | P21741 |
| 65 | 4016 | X | 13618-15 | MD1L1 | Mitotic spindle assembly | Q9Y6D9 |
| | | | | | checkpoint protein MAD1 | |
| 66 | 4053 | X | 9176-3 | MUC1:region 2 | Mucin-1:region 2 | P15941 |
| 67 | 4055 | X | 15565-102 | CA125 | Mucin-16 | Q8WXI7 |
| 68 | 4075 | X | 10362-35 | c-Myc | Myc proto-oncogene protein | P01106 |
| 69 | 4194 | X | 5734-13 | nectin-4 | Nectin-4 | Q96NY8 |
| 70 | 4341 | X | 5011-11 | PBEF | Nicotinamide phosphoribosyl- | P43490 |
| | | | | | transferase | |
| 71 | 4386 | X | 21995-20 | NOS | NOS | P29474 |
| 72 | 4483 | X | 14063-17 | OSM | Oncostatin-M | P13725 |
| 73 | 4485 | X | 10892-8 | OSMR | Oncostatin-M-specific receptor | Q99650 |
| | | | | | subunit beta | |
| 74 | 4511 | X | 13113-7 | Osteopontin | Osteopontin | P10451 |
| 75 | 4740 | X | 3389-7 | PCI | Plasma serine protease inhibitor | P05154 |
| 76 | 4783 | X | 9235-3 | PXDC1 | Plexin domain-containing | Q8IUK5 |
| | | | | | protein 1 | |
| 77 | 5254 | X | 14011-17 | S100A11 | Protein S100-A11 | P31949 |
| 78 | 5255 | X | 5852-6 | S100A12 | Protein S100-A12 | P80511 |
| 79 | 5263 | X | 9750-7 | S100A4 | Protein S100-A4 | P26447 |
| 80 | 5315 | X | 22001-23 | PADI2 | Protein-arginine deiminase type-2 | Q9Y2J8 |
| 81 | 5365 | X | 4154-57 | P-Selectin | P-selectin | P16109 |
| 82 | 5375 | X | 10672-75 | SP-B | Pulmonary surfactant-associated | P07988 |
| | | | | | protein B | |
| 83 | 5453 | X | 5481-16 | RASA1 | Ras GTPase-activating protein 1 | P20936 |
| 84 | 5592 | X | 3079-62 | TIG2 | Retinoic acid receptor responder | Q99969 |
| | | | | | protein 2 | |
| 85 | 6159 | X | 5496-49 | Spondin-1 | Spondin-1 | Q9HCB6 |
| 86 | 6163 | X | 21832-31 | SCCA1 | Squamous cell carcinoma antigen 1 | P29508 |
| 87 | 6191 | X | 2330-2 | SDF-1 | Stromal cell-derived factor 1 | P48061 |
| 88 | 6201 | X | 8479-4 | MMP-10 | Stromelysin-2 | P09238 |
| 89 | 6461 | X | 9233-71 | TFPI -2 | Tissue factor pathway inhibitor 2 | P48307 |
| 90 | 6471 | X | 3324-51 | LY9 | T-lymphocyte surface antigen Ly-9 | Q9HBG7 |
| 91 | 6547 | X | 18294-26 | SOX2 | Transcription factor SOX-2 | P48431 |
| 92 | 6753 | X | 3059-50 | BAFF | Tumor necrosis factor ligand | Q9Y275 |
| | | | | | superfamily member 13B | |
| 93 | 6761 | X | 3052-8 | Fas ligand, | Tumor necrosis factor ligand | P48023 |
| | | | | soluble | superfamily member 6, soluble form | |
| 94 | 6765 | X | 7693-13 | TRAIL R2 | Tumor necrosis factor receptor | O14763 |
| | | | | | superfamily member 10B | |
| 95 | 6770 | X | 8304-50 | OPG | Tumor necrosis factor receptor | O00300 |
| | | | | | superfamily member 11B | |
| 96 | 6797 | X | 5070-76 | DcR3 | Tumor necrosis factor receptor | O95407 |
| | | | | | superfamily member 6B | |
| 97 | 7068 | X | 2652-15 | suPAR | Urokinase plasminogen activator | Q03405 |
| | | | | | surface receptor | |
| 98 | 7104 | X | 2597-8 | VEGF | Vascular endothelial growth | P15692 |
| | | | | | factor A | |
| 99 | 7169 | X | 6385-63 | VWA1 | von Willebrand factor A domain- | Q6PCB0 |
| | | | | | containing protein 1 | |
| 100 | 7194 | X | 11388-75 | HE4 | WAP four-disulfide core domain | Q14508 |
| | | | | | protein 2 | |

| | | | Inflammation | | | | | |
|---|---|---|---|---|---|---|---|---|
| List of 100 | | Cardiovascular | and Immune | Metabolic | | | | |
| proteins | GeneID | Disease | Response | Disease | Oncology | Neuroscience | Cytokines | Respiratory |
| 1 | ADAMTS13 | X | X | | | | | |
| 2 | ADAM8 | | | | X | | | |
| 3 | ALDH3A1 | | X | | | X | | |
| 4 | ALPP | | | | | | | |
| 5 | FUT5 | | | | | | | |
| 6 | SERPINA1 | X | X | X | X | X | | X |
| 7 | AFP | | | X | X | | | |
| 8 | AREG | X | X | X | X | | X | X |
| 9 | ANGPT2 | | X | | | | | |
| 10 | ANXA1 | X | | | X | | | |
| 11 | RFNG | | | | X | | | |
| 12 | PPP3R1 | X | | | | | | |
| 13 | CASP8 | X | X | X | X | X | | X |
| 14 | CTSV | | | | | | | |
| 15 | CCL7 | X | X | | | X | | X |
| 16 | TP53 | X | X | X | X | X | | X |
| 17 | CHI3L1 | X | | | X | | | |
| 18 | CGB3 | | | | | | | |
| 19 | CD300E | | | | X | | | |
| 20 | CFHR5 | X | X | X | | | | |
| 21 | CRP | X | X | X | | X | | X |
| 22 | CLEC4D | | X | | | | | X |
| 23 | CDCP1 | | X | | | | | |
| 24 | CXCL13 | | X | | X | | | X |
| 25 | CXCL17 | | | | | | | |
| 26 | CXCL9 | X | X | X | X | X | X | X |
| 27 | CSTB | X | | | X | X | | |
| 28 | FCRLB | | | | | | | |
| 29 | FCRL5 | | | | | | | |
| 30 | FGF2 | X | X | X | X | X | X | X |
| 31 | FGF23 | X | X | X | | | | |
| 32 | FUT3 | | | | | | | |
| 33 | LGALS3BP | | | | | | | |
| 34 | ENO2 | | | | X | X | | X |
| 35 | GRP | | | | X | X | | |
| 36 | GSN | X | X | X | | X | | |
| 37 | PAEP | | | | X | | | |
| 38 | GDF15 | X | | | X | | X | |
| 39 | HGF | X | X | X | X | X | | X |
| 40 | HSP90AA1 | | X | | X | | | |
| 41 | FCAR | | | | | | | |
| 42 | KPNA1 | | X | | | | | |
| 43 | DPP10 | | X | | | | | |
| 44 | IGFBP1 | X | | | | | | |
| 45 | IGFBP2 | X | | X | | | | |
| 46 | ICAM1 | X | X | X | X | X | | X |
| 47 | IFNG | X | X | X | X | X | X | X |
| 48 | IL10RA | | | | | | | |
| 49 | IL2RA | X | X | X | | X | | X |
| 50 | IL6 | X | X | X | X | X | X | X |
| 51 | CXCL8 | X | X | X | X | X | | X |
| 52 | KRT19 | | X | X | | X | | X |
| 53 | KITLG | X | X | | X | X | X | X |
| 54 | SPINT1 | | | | | | | |
| 55 | LEP | X | X | X | X | X | | |
| 56 | LPL | X | X | X | X | | | |
| 57 | LYPD3 | | | | | | | |
| 58 | MMP12 | X | X | X | | X | | X |
| 59 | MMP7 | X | X | | X | | | |
| 60 | MMP9 | X | X | X | X | X | | X |
| 61 | MAGEA4 | | | | | | | |
| 62 | MSLN | | | | X | | | X |
| 63 | TIMP1 | X | X | X | | X | X | X |
| 64 | MDK | | | | X | X | | X |
| 65 | MAD1L1 | | X | | X | | | |
| 66 | MUC1 | X | X | | X | | | X |
| 67 | MUC16 | | X | | X | | | |
| 68 | MYC | X | X | | X | | | X |
| 69 | NECTIN4 | | | | | | | |
| 70 | NAMPT | | X | X | X | | X | |
| 71 | NOS3 | X | X | X | X | X | | X |
| 72 | OSM | | X | | | | X | |
| 73 | OSMR | X | | X | | | | X |
| 74 | SPP1 | X | X | X | X | X | X | X |
| 75 | SERPINA5 | X | | X | | | | |
| 76 | PLXDC1 | | | | | | | |
| 77 | S100A11 | | | | X | | | |
| 78 | S100A12 | | X | | | | | |
| 79 | S100A4 | X | | | X | X | | X |
| 80 | PADI2 | | | | | | | |
| 81 | SELP | X | X | | | X | | X |
| 82 | SFTPB | | | | | | | X |
| 83 | RASA1 | X | | | X | X | | |
| 84 | RARRES2 | X | | X | | | | |
| 85 | SPON1 | | | | | | | |
| 86 | SERPINB3 | | | | X | | | |
| 87 | CXCL12 | X | X | | X | | | X |
| 88 | MMP10 | | X | | X | | | X |
| 89 | TFPI2 | | X | | X | | | |
| 90 | LY9 | | | | | | | |
| 91 | SOX2 | | | | X | X | | X |
| 92 | TNFSF13B | X | | | | | X | |
| 93 | FASLG | X | X | X | X | X | X | X |
| 94 | TNFRSF10B | | | | | | | X |
| 95 | TNFRSF11B | X | X | X | | X | X | |
| 96 | TNFRSF6B | | | | | | | |
| 97 | PLAUR | X | X | | X | | | |
| 98 | VEGFA | X | X | | X | X | X | X |
| 99 | VWA1 | | | | | | | |
| 100 | WFDC2 | | | | X | | | |

TABLE 2
Top 6 individual proteins identified using cell-free DNA
fragmentomes and protein analysis of matched plasma

| # | Protein |
|---|---|
| 1 | MMP-12 |
| 2 | CRP |
| 3 | HE4 |
| 4 | IL-8 |
| 5 | FUT5 |
| 6 | S100A12 |

TABLE 3
Top influential proteins

| # | Protein |
|---|---|
| 1 | CA125 |
| 2 | CRP |
| 3 | IL-8 |
| 4 | MMP-12 |
| 5 | S100A12 |
| 6 | Midkine |
| 7 | MUC1: region 2 |
| 8 | CDCP1 |
| 9 | BLC |
| 10 | OSMR |
| 11 | FUT5 |
| 12 | HAI-1 |
| 13 | Fas ligand, soluble |
| 14 | MMP-9 |
| 15 | HSP 90a |
| 16 | OSM |
| 17 | PADI2 |

### Example 3: Detection of Lung Cancer Using Olink Proteins and Cell-Free DNA Fragmentomes

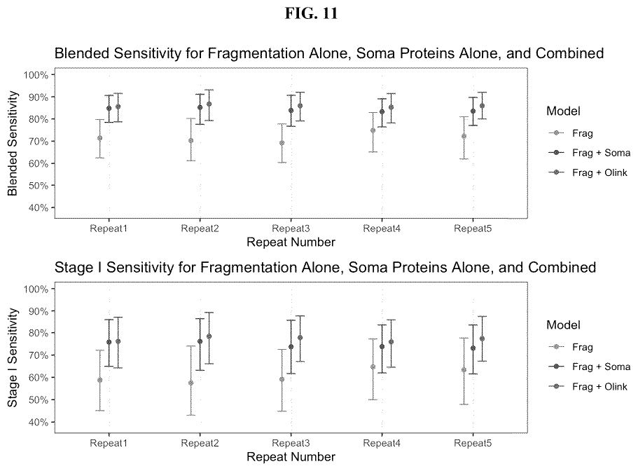

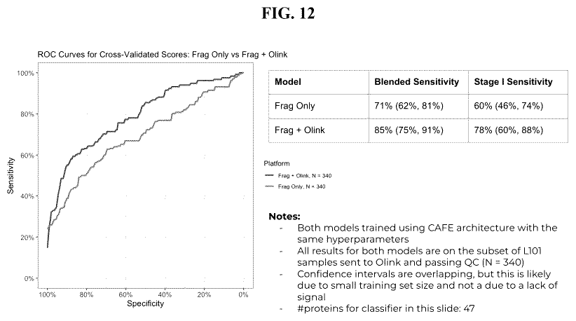

An initial evaluation of the utility of proteins was with SomaLogic measurements, which showed promise, but it was still necessary to do a preliminary analysis with the Olink protein panel to see whether the additive performance of Olink proteins is similar to what was observed in SomaLogic. The results of that analysis were that Olink provided a similar performance boost to that of SomaLogic, with both sets of analytes increasing blended sensitivity from ˜71% to ˜85% in a subset of L101 samples (N=340 samples and 86 proteins that overlapped SomaLogic's panel, Olink's Explore HT panel, and Delfi's internal literature-curated panel). The results of this analysis are shown in FIG. 11. The Olink Reveal panel allows the measurement of over 1000 proteins via NGS at a relatively low cost. The next analysis aimed to quantify the performance boost of proteins on the Olink Reveal panel, which is a subset of those on the Olink's Explore HT panel. The intersection of Delfi's internal literature-curated list and the Olink Reveal panel was 47 proteins shown in Table 5, so the next analysis aimed to characterize the performance boost of this smaller subset of proteins. The point estimate for the performance boost of 47 proteins on the Reveal-literature-overlap-panel is approximately the same as that observed in the 86 panels on the ExploreHT-literature-overlap-panel shown in Table 4. The results of this analysis are visualized in FIG. 12. For high-risk lung cancer screening, current approaches using cell-free DNA fragmentomes have been promising (Mazzone et al. 2024, Mathios et al. 2020). This study provides evidence that inclusion of an additional data modality (proteomics) boosts the performance of a classifier that already includes fragmentomics features. This study also shows the feasibility of a specific platform (Olink Reveal) in combination with fragmentomics. This study suggests the possibility of a liquid biopsy approach that has a superior performance profile compared to one using fragmentomics alone. The findings combined with the affordability of proteomic platforms such as Olink Reveal, could lead to a multi-omics approaches with improved outcomes for patients.

TABLE 4
ExploreHT-literature-overlap-panel

| Gene | UniProt |
|---|---|
| MAGEA4 | P43358 |
| IL10RA | Q13651 |
| IFNG | P01579 |
| FCRLB | Q6BAA4 |
| SOX2 | P48431 |
| NOS3 | P29474 |
| PADI2 | Q9Y2J8 |
| NAMPT | P43490 |
| RASA1 | P20936 |
| TP53 | P04637 |
| ALDH3A1 | P30838 |
| MAD1L1 | Q9Y6D9 |
| OSM | P13725 |
| PPP3R1 | P63098 |
| MUC16 | Q8WXI7 |
| KRT19 | P08727 |
| CASP8 | Q14790 |
| CCL7 | P80098 |
| VEGFA | P15692 |
| ANGPT2 | O15123 |
| HGF | P14210 |
| AREG | P15514 |
| FGF2 | P09038 |
| FASLG | P48023 |
| LY9 | Q9HBG7 |
| CTSV | O60911 |
| CXCL8 | P10145 |
| FGF23 | Q9GZV9 |
| MSLN | Q13421 |
| MMP12 | P39900 |
| IL6 | P05231 |
| FCAR | P24071 |
| TNFRSF6B | O95407 |
| S100A12 | P80511 |
| GRP | P07492 |
| VWA1 | Q6PCB0 |
| CDCP1 | Q9H5V8 |
| TNFRSF10B | O14763 |
| CLEC4D | Q8WXI8 |
| ALPP | P05187 |
| DPP10 | Q8N608 |
| CD300E | Q496F6 |
| PAEP | P09466 |
| CXCL17 | Q6UXB2 |
| ENO2 | P09104 |
| WFDC2 | Q14508 |
| LYPD3 | O95274 |
| CXCL13 | O43927 |
| S100A11 | P31949 |
| ADAM8 | P78325 |
| LPL | P06858 |
| PLAUR | Q03405 |
| MMP7 | P09237 |
| MDK | P21741 |
| ANXA1 | P04083 |
| SPON1 | Q9HCB6 |
| NECTIN4 | Q96NY8 |
| TNFRSF11B | O00300 |
| MMP10 | P09238 |
| LEP | P41159 |
| CXCL9 | Q07325 |
| TFPI2 | P48307 |
| KITLG | P21583 |
| SPP1 | P10451 |
| IGFBP1 | P08833 |
| CSTB | P04080 |
| IGFBP2 | P18065 |
| MMP9 | P14780 |
| SPINT1 | O43278 |
| TNFSF13B | Q9Y275 |
| IL2RA | P01589 |
| ADAMTS13 | Q76LX8 |
| GDF15 | Q99988 |
| AFP | P02771 |
| FCRL5 | Q96RD9 |
| MUC1 | P15941 |
| OSMR | Q99650 |
| CHI3L1 | P36222 |
| CGB3_CGB5_CGB8 | P0DN86 |
| TIMP1 | P01033 |
| RARRES2 | Q99969 |
| CFHR5 | Q9BXR6 |
| SELP | P16109 |
| ICAM1 | P05362 |
| SERPINA1 | P01009 |
| LGALS3BP | Q08380 |

TABLE 5
Reveal-literature-overlap-panel

| Gene | UniProt |
|---|---|
| ADAM8 | P78325 |
| CLEC5A | Q9NY25 |
| CXCL9 | Q07325 |
| KITLG | P21583 |
| LPL | P06858 |
| MMP10 | P09238 |
| S100A11 | P31949 |
| TNFRSF11B | O00300 |
| ALDH3A1 | P30838 |
| CASP8 | Q14790 |
| CCL7 | P80098 |
| CD300E | Q496F6 |
| CDCP1 | Q9H5V8 |
| CLEC4D | Q8WXI8 |
| CTSV | O60911 |
| CXCL17 | Q6UXB2 |
| CXCL8 | P10145 |
| DPP10 | Q8N608 |
| FASLG | P48023 |
| FCAR | P24071 |
| FGF2 | P09038 |
| FGF23 | Q9GZV9 |
| GRP | P07492 |
| HGF | P14210 |
| IL6 | P05231 |
| KRT19 | P08727 |
| LAMP3 | Q9UQV4 |
| LY9 | Q9HBG7 |
| MAD1L1 | Q9Y6D9 |
| MMP12 | P39900 |
| MSLN | Q13421 |
| MUC16 | Q8WXI7 |
| OSM | P13725 |
| PAEP | P09466 |
| S100A12 | P80511 |
| TNFRSF10B | O14763 |
| TNFRSF6B | O95407 |
| TNR | Q92752 |
| VEGFA | P15692 |
| VWA1 | Q6PCB0 |
| CEACAM5 | P06731 |
| IFNG | P01579 |
| IL10RA | Q13651 |
| NOS3 | P29474 |
| PADI2 | Q9Y2J8 |
| SFTPA2 | Q8IWL1 |
| TP53 | P04637 |

In certain embodiments and aspects, the present disclosure includes the following:

1. A diagnostic assay system, comprising:
a genomic component configured to:
   a) generate DNA sequences from input patient samples using a next-generation sequencing (NGS)-based assay workflow;
   b) associate DNA sequencing results with source patients using DNA-based barcodes; and
   c) process DNA sequencing results associated with each patient through a computer analysis pipeline;
a proteomic component configured to:
   a) perform a multiplexed protein detection assay with an NGS-based readout;
   b) multiplex a range of proteins from a handful to tens of thousands in a single sample;
   c) target specific protein content with a cocktail of chosen affinity binding molecules;
   d) associate NGS readout of protein assay results with source patients using DNA-based barcodes compatible with the genomic component; and
   e) process NGS readout of protein assay results associated with each patient through a computer analysis pipeline;
liquid handling robots configured to carry out one or more assay steps of the genomic and proteomic components;
a laboratory information management system (LIMS) configured to:
   a) track one or more assay steps;
   b) govern actions of the liquid handling robots;
   c) track and enforce the use of any protein-content specifying reagent at the appropriate point in the assay based on operator selection or a test requisition form; and
   d) track patient identities or patient-associated codes for samples and generate test information for both the proteomic and genomic components; and
a software classifier component configured to combine information generated by the genomic and proteomic components into a reported risk score for a patient for one or more types of cancer.

2. The diagnostic assay system of claim 1, further comprising a pooling feature configured to pool NGS libraries from the genomic and proteomic components to allow simultaneous readout of both components.

3. The diagnostic assay system of claim 1, wherein the proteomic component includes a modular protein content design, comprising two or more disease-specific associated protein reagents, enabling a laboratory to run multiple tests simultaneously on the same robot deck with each test having differences in protein reagent, classifier or both; and reporting among the different disease tests.

4. The diagnostic assay system of claim 1, wherein the proteomic component includes a universal protein content design, comprising: a single protein reagent containing all affinity binding molecules for all tests, with differentiation of employed content for different tests occurring informatically through filtering of sequences associated with specific proteins, followed by the use of disease-specific classifiers and reports.

5. A proteomic discovery system comprises:
the genomic component of the assay system of claim 1;
the proteomic component of the assay system of claim 1 using a large discovery panel of protein content;
one or more cohorts of patients known to have the disease or diseases in question; the running of the proteomic component of the assay system with a large discovery panel of protein content; and
a machine learning algorithm configured to generate a classifier that combines information generated by the genomic and proteomic components into a reported risk score for a patient for the disease or diseases in question.

6. The diagnostic assay system of claim 1, wherein the proteomic component is further configured to allow for the discovery and efficient deployment of integrated genomic and proteomic diagnostic assays, enabling efficient discovery and modular deployment of protein-based panels in the context of a genomic-based workflow.

7. A method for detecting lung cancer in an individual, comprising:
a) analyzing a sample obtained from the individual to detect a presence of a panel of proteins using a protein platform;
b) assessing cell-free DNA fragmentation patterns in the sample;
c) applying a machine learning model to the detected proteins and cell-free DNA fragmentation patterns to generate an area under the curve (AUC) score; and
d) determining the presence of lung cancer in the individual based on the AUC score.

8. The method of claim 7, wherein the sample is a L101 sample.

9. The method of claim 7, wherein the machine learning model includes a gradient boosting machine (GBM) model.

10. The method of claim 7, wherein the AUC score for the combined analysis of proteins and cell-free DNA fragmentation patterns is at least about 0.90.

11. The method of claim 7, wherein the AUC score for stage I lung cancer is at least about 0.81.

12. The method of claim 7, further comprising:
a) evaluating the performance of the combined protein and cell-free DNA fragmentation model at 50% specificity; and
b) determining the sensitivity for detecting stage I, stage II, and stage III & IV lung cancer.

13. The method of claim 12, wherein the sensitivity for detecting stage I lung cancer is at least about 88%.

14. The method of claim 12, wherein the sensitivity for detecting stage II lung cancer is at least about 96%.

15. The method of claim 12, wherein the sensitivity for detecting stage III & IV lung cancer is about 100%.

16. A system for detecting lung cancer in an individual, comprising:
a) a protein platform configured to analyze a sample from the individual to detect a presence of a panel of proteins;
b) a cell-free DNA fragmentation analysis module configured to assess cell-free DNA fragmentation patterns in the sample;
c) a machine learning module configured to apply a machine learning model to the detected proteins and cell-free DNA fragmentation patterns to generate an AUC score; and
d) a diagnostic module configured to determine the presence of lung cancer in the individual based on the AUC score.

17. The system of claim 16, wherein the machine learning module includes a gradient boosting machine (GBM) model.

18. The system of claim 16, wherein the diagnostic module is further configured to evaluate the performance of the combined protein and cell-free DNA fragmentation model at about 50% specificity and to determine the sensitivity for detecting stage I, stage II, and stage III & IV lung cancer.

19. A non-transitory computer-readable medium storing instructions that, when executed by a processor, cause the processor to perform a method for detecting lung cancer in an individual, the method comprising:
a) receiving data indicative of a presence of a panel of proteins in a sample from the individual, wherein the proteins are analyzed using a protein platform;
b) receiving data indicative of cell-free DNA fragmentation patterns in the sample;
c) applying a machine learning model to the received data to generate an AUC score; and
d) outputting a determination of the presence of lung cancer in the individual based on the AUC score.

20. The non-transitory computer-readable medium of claim 19, wherein the machine learning model includes a gradient boosting machine (GBM) model.

21. The non-transitory computer-readable medium of claim 19, wherein the method further comprises instructions for evaluating the performance of the combined protein and cell-free DNA fragmentation model at about 50% specificity and for determining the sensitivity for detecting stage I, stage II, and stage III & IV lung cancer.

22. A method for detecting lung cancer in an individual, comprising:
a) measuring levels of a panel of literature-curated proteins in a sample from the individual using a protein platform;
b) analyzing cell-free DNA fragmentation patterns in the sample;
c) applying a machine learning model to the measured levels of the proteins and the analyzed cell-free DNA fragmentation patterns to determine a combined area under the curve (AUC) score; and
d) diagnosing the presence or stage of lung cancer in the individual based on the combined AUC score.

23. The method of claim 22, wherein the sample is a L101 sample.

24. The method of claim 22, wherein the machine learning model is a gradient boosting machine (GBM) model.

25. The method of claim 22, wherein the panel of proteins is associated with lung cancer risk.

26. The method of claim 22, wherein the machine learning model provides a combined AUC of about 0.86 (0.82-0.9) for the proteins.

27. The method of claim 22, wherein the combined AUC for stage I lung cancer is about 0.75 (0.68-0.82) when using the proteins alone.

28. The method of claim 22, wherein the combined AUC for detecting lung cancer using both proteins and cell-free DNA fragmentation is about 0.90 (0.87-0.93).

29. The method of claim 22, wherein the combined AUC for stage I lung cancer using both proteins and cell-free DNA fragmentation is about 0.81 (0.75-0.88).

30. The method of claim 22, further comprising evaluating the performance of the combined protein and cell-free DNA fragmentation model at about 50% specificity to determine sensitivities for different stages of lung cancer.

31. The method of claim 22, wherein the sensitivities at about 50% specificity are about 88% for stage I, about 96% for stage II, and about 100% for stages III & IV.

32. The method of claim 22, further comprising identifying a subset of proteins from the panel of proteins that contribute to detection benefit, wherein the subset comprises about 20 or fewer proteins.

33. The method of claim 22, wherein the identification of the subset of proteins is performed using an iterative process that removes the least influential protein in each iteration.

34. The method of claim 22, wherein the iterative process results in a list of top influential proteins that maximizes performance and lowers the potential cost of the combined assay.

35. A system for detecting lung cancer in an individual, comprising:
a) a protein platform configured to measure levels of a panel of literature-curated proteins in a sample from the individual;
b) an analyzer configured to analyze cell-free DNA fragmentation patterns in the sample;
c) a processor configured to apply a machine learning model to the measured levels of the proteins and the analyzed cell-free DNA fragmentation patterns to determine a combined AUC score; and
d) a diagnostic module configured to diagnose the presence or stage of lung cancer in the individual based on the combined AUC score.

36. A non-transitory computer-readable medium containing instructions that, when executed by a processor, perform a method for detecting lung cancer in an individual, the method comprising:
a) receiving data corresponding to levels of a panel of literature-curated proteins measured in a sample from the individual;
b) receiving data corresponding to cell-free DNA fragmentation patterns analyzed in the sample;
c) applying a machine learning model to the received data to determine a combined AUC score; and
d) outputting a diagnosis for the presence or stage of lung cancer in the individual based on the combined AUC score.

37. The method of claim 7, wherein the method comprises detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof.

38. The method of claim 37, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, or TP53 or any combination thereof.

39. The system of claim 16, wherein the method at a) comprises detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof.

40. The system of claim 39, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

41. The method of claim 19, wherein the method at a) comprises detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof.

42. The method of claim 41, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53, or any combination thereof.

43. The method of claim 22, wherein the method at a) comprises detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3 CGB5 CGB8, TIMP1, RARRES2, CFHR5, SELP, ICAM1, SERPINA1, LGALS3BP or any combination thereof.

44. The method of claim 43, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

45. The method of claim 35, wherein the method at a) comprises detecting the presence of a panel of proteins comprising MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof.

46. The method of claim 45, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

Although the invention has been described with reference to the presently preferred embodiment, it should be understood that various modifications can be made without departing from the spirit of the invention. Accordingly, the invention is limited only by the following claims.

What is claimed is:

1. A diagnostic assay system, comprising:
a genomic component configured to:
a) generate DNA sequences from input patient samples using a next-generation sequencing (NGS)-based assay workflow;
b) associate DNA sequencing results with source patients using DNA-based barcodes; and
c) process DNA sequencing results associated with each patient through a computer analysis pipeline;
a proteomic component configured to:
a) perform a multiplexed protein detection assay with an NGS-based readout;
b) multiplex a range of proteins from a handful to tens of thousands in a single sample;
c) target specific protein content with a cocktail of chosen affinity binding molecules;
d) associate NGS readout of protein assay results with source patients using DNA-based barcodes compatible with the genomic component; and
e) process NGS readout of protein assay results associated with each patient through a computer analysis pipeline;
liquid handling robots configured to carry out one or more assay steps of the genomic and proteomic components;
a laboratory information management system (LIMS) configured to:
a) track one or more assay steps;
b) govern actions of the liquid handling robots;
c) track and enforce the use of any protein-content specifying reagent at the appropriate point in the assay based on operator selection or a test requisition form; and
d) track patient identities or patient-associated codes for samples and generate test information for both the proteomic and genomic components; and
a software classifier component configured to combine information generated by the genomic and proteomic components into a reported risk score for a patient for one or more types of cancer.

2. The diagnostic assay system of claim 1, further comprising a pooling feature configured to pool NGS libraries from the genomic and proteomic components to allow simultaneous readout of both components.

3. The diagnostic assay system of claim 1, wherein the proteomic component includes a modular protein content design, comprising two or more disease-specific associated protein reagents, enabling a laboratory to run multiple tests simultaneously on the same robot deck with each test having differences in protein reagent, classifier, or both; and reporting among the different disease tests.

4. The diagnostic assay system of claim 1, wherein the proteomic component includes a universal protein content design, comprising: a single protein reagent containing all affinity binding molecules for all tests, with differentiation of employed content for different tests occurring informatically through filtering of sequences associated with specific proteins, followed by the use of disease-specific classifiers and reports.

5. A proteomic discovery system comprises:
the genomic component of the assay system of claim 1;
the proteomic component of the assay system of claim 1 using a large discovery panel of protein content;
one or more cohorts of patients known to have the disease or diseases in question;
the running of the proteomic component of the assay system with a large discovery panel of protein content; and
a machine learning algorithm configured to generate a classifier that combines information generated by the genomic and proteomic components into a reported risk score for a patient for the disease or diseases in question.

6. The diagnostic assay system of claim 1, wherein the proteomic component is further configured to allow for the discovery and efficient deployment of integrated genomic and proteomic diagnostic assays, enabling efficient discovery and modular deployment of protein-based panels in the context of a genomic-based workflow.

7. A method for detecting lung cancer in an individual, comprising:
a) analyzing a sample obtained from the individual to detect a presence of a panel of proteins using a protein platform;
b) assessing cell-free DNA fragmentation patterns in the sample;
c) applying a machine learning model to the detected proteins and cell-free DNA fragmentation patterns to generate an area under the curve (AUC) score; and
d) determining the presence of lung cancer in the individual based on the AUC score.

8. The method of claim 7, wherein the sample is a L101 sample.

9. The method of claim 7, wherein the machine learning model includes a gradient boosting machine (GBM) model.

10. The method of claim 7, wherein the AUC score for the combined analysis of proteins and cell-free DNA fragmentation patterns is at least about 0.90.

11. The method of claim 7, wherein the AUC score for stage I lung cancer is at least about 0.81.

12. The method of claim 7, further comprising:
a) evaluating the performance of the combined protein and cell-free DNA fragmentation model at 50% specificity; and
b) determining the sensitivity for detecting stage I, stage II, and stage III & IV lung cancer.

13. The method of claim 12, wherein the sensitivity for detecting stage I lung cancer is at least about 88%.

14. A system for detecting lung cancer in an individual, comprising:
a) a protein platform configured to analyze a sample from the individual to detect a presence of a panel of proteins;
b) a cell-free DNA fragmentation analysis module configured to assess cell-free DNA fragmentation patterns in the sample;
c) a machine learning module configured to apply a machine learning model to the detected proteins and cell-free DNA fragmentation patterns to generate an AUC score; and
d) a diagnostic module configured to determine the presence of lung cancer in the individual based on the AUC score.

15. The system of claim 14, wherein the machine learning module includes a gradient boosting machine (GBM) model.

16. The system of claim 14, wherein the diagnostic module is further configured to evaluate the performance of the combined protein and cell-free DNA fragmentation model at about 50% specificity and to determine the sensitivity for detecting stage I, stage II, and stage III & IV lung cancer.

17. A method for detecting lung cancer in an individual, comprising:
a) measuring levels of a panel of literature-curated proteins in a sample from the individual using a protein platform;
b) analyzing cell-free DNA fragmentation patterns in the sample;
c) applying a machine learning model to the measured levels of the proteins and the analyzed cell-free DNA fragmentation patterns to determine a combined area under the curve (AUC) score; and
d) diagnosing the presence or stage of lung cancer in the individual based on the combined AUC score.

18. The method of claim 17, wherein the sample is a L101 sample.

19. The method of claim 17, wherein the machine learning model is a gradient boosting machine (GBM) model.

20. The method of claim 17, wherein the panel of proteins is associated with lung cancer risk.

21. The method of claim 17, wherein the machine learning model provides a combined AUC of about 0.86 (0.82-0.9) for the proteins.

22. The method of claim 17, wherein the combined AUC for stage I lung cancer is about 0.75 (0.68-0.82) when using the proteins alone.

23. The method of claim 17, wherein the combined AUC for detecting lung cancer using both proteins and cell-free DNA fragmentation is about 0.90 (0.87-0.93).

24. The method of claim 17, wherein the combined AUC for stage I lung cancer using both proteins and cell-free DNA fragmentation is about 0.81 (0.75-0.88).

25. The method of claim 17, further comprising evaluating the performance of the combined protein and cell-free DNA fragmentation model at about 50% specificity to determine sensitivities for different stages of lung cancer.

26. The method of claim 17, wherein the sensitivities at about 50% specificity are about 88% for stage I, about 96% for stage II, and about 100% for stages III & IV.

27. The method of claim 17, wherein the identification of the subset of proteins is performed using an iterative process that removes the least influential protein in each iteration.

28. The method of claim 17, wherein the iterative process results in a list of top influential proteins that maximizes performance and lowers the potential cost of the combined assay.

29. A system for detecting lung cancer in an individual, comprising:
a) a protein platform configured to measure levels of a panel of literature-curated proteins in a sample from the individual;
b) an analyzer configured to analyze cell-free DNA fragmentation patterns in the sample;
c) a processor configured to apply a machine learning model to the measured levels of the proteins and the analyzed cell-free DNA fragmentation patterns to determine a combined AUC score; and
d) a diagnostic module configured to diagnose the presence or stage of lung cancer in the individual based on the combined AUC score.

30. The method of claim 7, wherein the panel of proteins comprises MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof.

31. The method of claim 30, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, or TP53 or any combination thereof.

32. The system of claim 29, wherein the panel of proteins comprises MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3_CGB5_CGB8, TIMP1, RARRES2, CFHR5, SELP, ICAM1, SERPINA1, LGALS3BP or any combination thereof.

33. The system of claim 32, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, TP53 or any combination thereof.

34. The method of claim 17, wherein the panel of proteins comprises MAGEA4, IL10RA, IFNG, FCRLB, SOX2, NOS3, PADI2, NAMPT, RASA1, TP53, ALDH3A1, MAD1L1, OSM, PPP3R1, MUC16, KRT19, CASP8, CCL7, VEGFA, ANGPT2, HGF, AREG, FGF2, FASLG, LY9, CTSV, CXCL8, FGF23, MSLN, MMP12, IL6, FCAR, TNFRSF6B, S100A12, GRP, VWA1, CDCP1, TNFRSF10B, CLEC4D, ALPP, DPP10, CD300E, PAEP, CXCL17, ENO2, WFDC2, LYPD3, CXCL13, S100A11, ADAM8, LPL, PLAUR, MMP7, MDK, ANXA1, SPON1, NECTIN4, TNFRSF11B, MMP10, LEP, CXCL9, TFPI2, KITLG, SPP1, IGFBP1, CSTB, IGFBP2, MMP9, SPINT1, TNFSF13B, IL2RA, ADAMTS13, GDF15, AFP, FCRL5, MUC1, OSMR, CHI3L1, CGB3 CGB5 CGB8, TIMP1, RARRES2, CFHR5, SELP, ICAM1, SERPINA1, LGALS3BP or any combination thereof.

35. The method of claim 34, comprising detecting the presence of a panel of proteins comprising ADAM8, CLEC5A, CXCL9, KITLG, LPL, MMP10, S100A11, TNFRSF11B, ALDH3A1, CASP8, CCL7, CD300E, CDCP1, CLEC4D, CTSV, CXCL17, CXCL8, DPP10, FASLG, FCAR, FGF2, FGF23, GRP, HGF, IL6, KRT19, LAMP3, LY9, MAD1L1, MMP12, MSLN, MUC16, OSM, PAEP, S100A12, TNFRSF10B, TNFRSF6B, TNR, VEGFA, VWA1, CEACAM5, IFNG, IL10RA, NOS3, PADI2, SFTPA2, or TP53 or any combination thereof.
